(19) United States
(12) Patent Application Publication
(10) Pub. No.: US 2008/0241862 A1
(43) Pub. Date: Oct. 2, 2008

(54) PROPIONYL AND BUTYRYL LYSINE MODIFICATIONS IN PROTEINS

(76) Inventors: YINGMING ZHAO, Dallas, TX (US); John Falck, University Park, TX (US); Yue Chen, Irving, TX (US)

Correspondence Address: FULBRIGHT & JAWORSKI L.L.P., 600 CONGRESS AVE., SUITE 2400, AUSTIN, TX, 78701, US

(21) Appl. No.: 12/021,960

(22) Filed: Jan. 29, 2008

Related U.S. Application Data
(60) Provisional application No. 60/897,993, filed on Jan. 29, 2007.

(51) Int. Cl. G01N 33/574; G01N 33/566; C07K 14/00; C12P 13/08; C07K 16/18; G01N 33/53

(52) U.S. Cl. 435/7.23; 436/501; 435/7.21; 435/272; 530/387.1; 435/115

(57) ABSTRACT

While the identification of acetylated lysine residues on proteins is well-known, the modification of lysine residues through propionylation and butyrylation is not very well understood. A method for the identification and mapping of propionylated and butyrylated lysine residues has been developed. Anti-acetyllysine antibody, normally used to affinity purify a protein mixture based on the presence of acetylated lysine, can also be used to affinity purity proteins having propionylated and butyrylated lysine residues due to the structural similarity. The method involves searching protein databases to locate mass spectrometry datasets for those proteins purified by anti-acetyllysine antibody. The located spectra are manually reviewed to identify those peptides having propionyllysine and butyryllysine residues. These identified peptides are synthesized, with the lysine modifications added at the appropriate positions. The synthesized proteins are then analyzed with mass spectrometry and the resultant spectra are compared to those located in the protein databases to confirm the location of the lysine modifications.

This application claims priority to U.S. Application No. 60/897,993, filed on Jan. 29, 2007, the entire disclosure of which is incorporated by reference.

### BACKGROUND

This invention pertains to the identification and mapping of modified lysine residues in proteins, and particularly to the identification of propionylated lysine and butyrylated lysine residues.

Molecular anatomy of post-translational modifications that regulate cellular processes and disease progression stands as one of the major goals of post-genomic biological research. To date, more than 200 post-translational modifications have been described, which provides an efficient way to diversify a protein's primary structure and possibly its functions. The remarkable complexity of these molecular networks is exemplified by modifications at the side chain of lysine, one of the fifteen ribosomally-coded amino acid residues known to be modified. The electron-rich and nucleophilic nature of the lysine side chain makes it suitable for undergoing covalent post-translational modification reactions with diverse substrates that are electrophilic. The residue can be potentially modulated by several post-translational modifications including methylation, acetylation, biotinylation, ubiquitination, and sumoylation, which have pivotal roles in cell physiology and pathology.

Histones are known to be modified by an array of post-translational modifications, including methylation, acetylation, ubiquitination, small ubiquitin-like modification, and ribosylation. A combinatorial array of post-translational modifications in histones, termed the “histone code”, dictates the proteins' functions in gene expression and chromatin dynamics. Post-translational modifications of histones have been studied by both biochemistry (Jenuwein, et al. 2001) and mass spectrometry (Garcia, et al. 2007; Boyne, et al. 2006; Medzihradszky, et al. 2004).

Lysine acetylation is an abundant, reversible, and highly regulated post-translational modification. While initially discovered in histones, the modification was later identified in non-histone proteins, such as p53. A recent proteomics screening showed that acetyllysine is abundant and present in substrates that are affiliated with multiple organelles and have diverse functions. Interestingly, the modification is enriched in mitochondrial proteins and metabolic enzymes, implying its roles in fine-tuning the organelle's functions and energy metabolism. The modification plays an important roles in diverse cellular processes, such as apoptosis, metabolism, transcription, and stress response. In addition to their roles in fundamental biology, lysine acetylation and its regulatory enzymes (acetyltransferases and deacetylases) are intimately linked to aging and several major diseases such as cancer, neurodegenerative disorders, and cardiovascular diseases.

Acetyl-CoA, a member of high-energy CoA compounds, is the substrate used by acetyltransferases to catalyze the lysine-acetylation reaction. It remains unknown, however, if cells could use other short-chain CoAs to carry out similar post-translational modifications at the lysine residue. No current reagent exists for the detection of certain of these modifications.

### SUMMARY OF THE INVENTION

In one embodiment, the present invention provides a method for detecting a propionyllysine or butyryllysine in a polypeptide comprising: (a) obtaining a sample comprising polypeptides; (b) separating the polypeptides by molecular weight; (c) contacting one or more of the separated polypeptides with an antibody that specifically binds with a polypeptide having a propionyllysine or butyryllysine, but does not substantially bind with a polypeptide that does not have a propionyllysine or butyryllysine; and (d) detecting the binding of the antibody to the polypeptides, whereby antibody binding to the polypeptides indicates the presence of the propionyllysine or butyryllysine in the polypeptides.

In another embodiment, the present invention provides a method for isolating a group of propionylated or butyrylated peptides from a complex mixture of peptides comprising: (a) digesting a proteinaceous material with a proteolytic enzyme or chemical cleavage agent to obtain digested proteinaceous material; (b) contacting the digested proteinaceous material with an immobilized propionyllysine-specific or butyryllysine-specific antibody; and (d) isolating from the digested proteinaceous material the target group of propionylated or butyrylated peptides specifically bound by the immobilized propionyllysine-specific or butyryllysine-specific antibody.

In a further embodiment, the present invention provides a method for detecting changes in propionylation and/or butyrylation of proteins associated with a disease state or a treatment comprising: (a) obtaining a first sample corresponding to a first disease state or a first treatment; (b) obtaining a second sample corresponding to a second disease state or a second treatment; (c) contacting the first sample and the second sample with an antibody that specifically binds with a polypeptide having a propionyllysine or butyryllysine, but does not substantially bind with a polypeptide that does not have a propionyllysine or butyryllysine; (d) detecting the specific binding of the antibody to the polypeptides in the samples, whereby antibody binding to the polypeptides indicates the presence of the propionyllysine or butyryllysine in the polypeptides; and (e) comparing the propionyllysine or butyryllysine modifications in the first sample with the propionyllysine or butyryllysine modifications in the second sample to identify changes in the propionyllysine or butyryllysine modifications associated with the disease or treatment. In certain embodiments the first disease state is the presence of disease, and the second disease state is the absence of the disease. In certain embodiments, the first treatment is treatment with a test compound, and the second treatment is a mock or placebo treatment. The comparison of the first and second samples may comprise quantification and/or characterization of the propionyllysine or butyryllysine modifications in a single polypeptide or in a group of polypeptides. The first and second samples may be digested, and the digested samples contacted with a propionyllysine-specific or butyryllysine-specific antibody. It is contemplated that either the propionylation or butyrylation, or both the propionylation and butyrylation of the polypeptides may be assayed.

The sample may be any sample containing, or suspected of containing proteinacious material (e.g., peptides, polypeptides, and/or proteins). In certain aspects of the invention the sample is obtained from a cell culture, tissue biopsy, or a clinical fluid. Non-limiting examples of clinical fluids include blood, serum, urine, saliva, synovial fluid, lymph fluid, and spinal fluid. In some embodiments the sample is digested (such as by proteolytic or chemical cleavage) to obtain a digested sample. The term “polypeptide” refers to a compound of a single chain or a complex of two or more chains of amino acid residues linked by peptide bonds. The chain(s) may be of any length. A protein is a polypeptide and the terms are used interchangeably herein. The term “peptide” is used herein to refer to a polypeptide of less than about 50 amino acids.

In certain embodiments of the invention the state of propionyl and/or butyryl modification of lysine residues in peptides, polypeptides, and/or proteins in a sample may be compared to the state of propionyl and/or butyryl modification of lysine residues in peptides, polypeptides, and/or proteins in a reference sample. The reference sample will preferably be of the same type as the “test” sample. For example, if the test sample is a serum sample then the reference sample is preferably also a serum sample. In certain embodiments, the state (e.g., presence or absence) of propionyl and/or butyryl modification of lysine residues in peptides, polypeptides, and/or proteins a test sample is compared with a reference sample from a “normal” (i.e., not diseased) organism or a diseased organism. In other embodiments, the methods for comprise comparing protein activation in the test sample with protein activation in the reference sample. In one embodiment, the disease is cancer. In other embodiments, the sample is treated or is obtained from an organism that was treated with at least one test compound and the reference sample is untreated and is obtained from an untreated organism. Alternatively, the sample is untreated and is obtained from an untreated organism and the reference sample is treated or is obtained from an organism that was treated with at least one test compound. In one embodiment, the test compound is a cancer therapeutic. By comparing the propionyl and/or butyryl modification of lysine residues among such sample it is possible to identify those modifications that are associated with, for example, a disease state, protein activation, or response to therapy. This information may then be used in, for example, disease diagnosis and decision making regarding the choice of therapy for disease treatment.

In certain aspects of the invention, the methods comprise separating polypeptides by molecular weight. In this manner, groups of proteins having the same or similar molecular weights are obtained. A variety of techniques for separating polypeptides by molecular weight are known in the art. One such technique is gel electrophoresis. Separation of polypeptides may be further enhanced by heating the sample to a temperature sufficient to denature the polypeptides, but not so high as to cause significant degradation of peptide bonds of the polypeptides. In certain aspects of the invention, the sample is treated with an enzyme inhibitor. Treatment with an enzyme is generally performed during sample preparation. If the sample is to be heat denatured, then the treatment with an enzyme inhibitor will typically be performed prior to heating. Examples of enzyme inhibitors include, but are not limited to, aprotinin (Trasylol™), phenylmethylsulfonyl fluoride (PMSF), benzamidine, diisopropylfluorophosphate (DIFP), leupeptin, pepstatin, EDTA, EGTA, sodium butyrate, trichostatin A, suberoylanilide hydroxamic acid (SAHA), FK288, nicotinamide, and sirtinol.

In certain aspects of the invention, polypeptides and/or antibodies may be immobilized on a solid support, such as on a resin, bead, chip, or nitrocellulose paper. In one embodiment, one or more of the polypeptides in a sample are immobilized on a solid support prior to contacting the polypeptides with an antibody. In another embodiment, one or more antibodies are immobilized on a solid support prior to contacting the polypeptides with the antibodies. In some embodiments, an antibody is covalently linked to a chromatography resin or noncovalently linked to protein-A- or protein-G-agarose. The resin may be contained, for example, within a column or micropipette tip.

The propionyl and butyryl modification of lysine residues in peptides, polypeptides, and/or proteins is detected using antibodies that specifically bind propionyllysine or butyryllysine. In certain embodiments, the binding specificity depends only on the presence of propionyllysine or butyryllysine and is independent of adjacent sequences. In other embodiments, the antibody's binding specificities may depend also on the protein sequence surrounding the propionyllysine or butyryllysine residue. For example, the antibody may bind an epitope containing a propionyllysine on a particular protein, but does not bind to a propionyllysine on a different protein nor does it bind to an identical epitope in which the lysine is not propionylated. Specific binding refers to a precise interaction between two molecules which is dependent upon their structure, such as the binding between an epitope of a protein and an antibody. Thus, an antibody that specifically binds propionyllysine or an epitope containing propionyllysine does not substantially bind to an unmodified lysine or an epitope that does not contain propionyllysine. Likewise, an antibody that specifically binds butyryllysine or an epitope containing butyryllysine does not substantially bind to an unmodified lysine or an epitope that does not contain butyryllysine. Antibodies that specifically bind propionyllysine or butyryllysine also do not substantially bind lysines with modifications other than propionylation or butyrylation (e.g., acetyllysine). An antibody that “does not substantially bind” to a particular epitope refers to an amount of binding between the molecules that is low enough so as not to interfere with a meaningful assay conducted to detect specific binding of the antibody to its intended epitope under a particular set of assay conditions. In one aspect, antibody is substantially incapable of binding or recognizing another molecule (cross-reacting) where the antibody exhibits a reactivity for the cross-reacting molecule that is less than 25%, preferably less than 10%, more preferably less than 5% of the reactivity exhibited toward the intended molecule under a particular set of assay conditions, which includes the relative concentration and incubation time of the molecules. Specific binding and cross-reactivity can be evaluated using a number of widely known methods, such as an immunohistochemical assay, an enzyme-linked immunosorbent assay (ELISA), a radioimmunoassay (RIA), or a Western blot assay.

Detecting the binding of an antibody to a polypeptide may be performed using a number of widely known methods, such as the immunohistochemical assay, enzyme-linked immunosorbent assay (ELISA), radioimmunoassay (RIA), or Western blot assay mentioned above. Detection is facilitated with the use of a label such as a radioactive label, fluorescent label, or chemiluminescent label. For example, a label may be attached directly to the antibody or it may be attached to a secondary antibody.

In some embodiments, the methods of the present invention comprises quantifying and/or characterizing the propionylated and/or butyrylated peptides. Such quantification and characterization may be performed using techniques such as mass spectrometry (MS), tandem mass spectrometry (MS/MS), MS3 analysis, or a combination thereof. In a particular embodiment, the quantification and/or characterization is performed using one or more of matrix-assisted laser desorption time-of-flight (MALDI-TOF) MS, liquid chromatography (LC)-MS/MS, or LC-MS3. Where the antibody is immobilized in a chromatography resin within a column, the column may be coupled to a mass spectrometer. In certain embodiments, the propionylated or butyrylated peptides may be quantified using stable isotope labeling by amino acids in cell culture (SILAC), isotope-coded affinity tag (ICAT), iTRAQ™ (Applied Biosystems), and/or absolute quantification of peptides (AQUA) techniques. The quantification and characterization may comprise comparing the propionyllysine and/or butyryllysine modifications of at least one of the propionylated or butyrylated polypeptides or peptides with the propionyllysine and/or butyryllysine modifications of a corresponding polypeptide or peptide in a reference sample.

As mentioned above, certain embodiments of the invention employ proteolytic enzymes or chemical cleavage agents to create digested proteinacious material. The proteolytic enzyme and chemical cleavage agents may be immobilized or used in solution. It is generally desirable to remove any proteolytic enzymes or chemical cleavage agents from the digested proteinacious material prior to contacting the digested material with antibodies to prevent the degradation of the antibodies. Where the proteolytic enzymes or chemical cleavage agents are immobilized on a solid support they may be physically separated from the digested material. Treatment with a proteolysis inhibitor may also be used prior to contacting the digested proteinaceous material with an antibody.

In one embodiment, the present invention provides an isolated antibody that specifically binds to a propionylated lysine or butyrylated lysine. In certain embodiments, the antibody that specifically binds to a propionylated lysine or butyrylated lysine does not substantially bind to acetylated lysine and unmodified lysine. The antibody may specifically bind to a propionylated lysine or butyrylated lysine or it may specifically bind to an epitope comprising propionylated lysine or butyrylated lysine. An antibody that specifically binds to an epitope comprising propionylated lysine or butyrylated lysine does not substantially bind to an epitope having an identical amino acid sequence but in which the lysine is not propionylated or butyrylated. In certain aspects of the invention, the isolated antibody specifically binds to propionylated lysine. In another aspect of the invention, the isolated antibody specifically binds to butyrylated lysine. In one embodiment, the isolated antibody specifically binds to both propionylated lysine and butyrylated lysine.

In another embodiment, the isolated antibody specifically binds to a propionylated lysine or butyrylated lysine in a histone H2B, H3, or H4 protein. In one embodiment, the present invention provides an isolated antibody that specifically binds to a propionylated lysine or butyrylated lysine at the sixth lysine residue from the amino terminus of a human histone H2B protein (lysine 20 according to the numbering in FIG. 19). In another embodiment, the present invention provides an isolated antibody that specifically binds to a propionylated lysine or butyrylated lysine at the third or fifth lysine residue from the amino terminus of a human histone H3 protein (lysines 14 and 23 according to the numbering in FIG. 19). In yet another embodiment, the present invention provides as isolated antibody that specifically binds to a propionylated lysine or butyrylated lysine at the first, third, or fourth lysine residue from the amino terminus of a human histone H4 (lysines 5, 8, 12, 16, 31, 44, 77, 79, and 91 according to the numbering in FIG. 19).

In another embodiment, the present invention provides an isolated antibody that specifically binds to a propionylated lysine or butyrylated lysine in a p53 protein. In yet another embodiment, the present invention provides an isolated antibody that specifically binds to a propionylated lysine or butyrylated lysine in a p300 protein. In a further embodiment, the present invention provides an isolated antibody that specifically binds to a propionylated lysine or butyrylated lysine in a CREB-binding protein (CBP).

In one embodiment, the present invention provides a method for identifying lysine-propionylated and lysine-butyrylated peptides in a peptide mixture, comprising: purifying the peptide mixture using affinity purification with anti-acetyllysine antibody.

In another embodiment, the present invention provides a method for in vitro propionylation or butyrylation of lysine residues in a protein comprising: incubating the protein with a purified acetyltransferase enzyme and propionyl-CoA, butyryl-CoA, or a mixture of both. The protein may be, for example, a core histone or p53. The purified acetyltransferase enzyme may be CBP or p300.

The antibodies disclosed herein may be provided in kits. Such kits will comprise one or more containers for holding antibodies as well as other reagents such as buffers or controls. In another embodiment, the present invention provides a kit for in vitro propionylation or butyrylation of lysine residues in a protein. Such a kit may comprise one or more of an acetyltransferase enzyme, propionyl-CoA, and/or butyryl-CoA. The components may be provided in separate containers within the kit or one or more of the components may be combined in a single container.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows the structures of acetyl CoA, propionyl CoA, and butyryl CoA, as well as the modified lysines acetyl lysine, propionyl lysine, and butyryl lysine;

FIG. 2 shows the tandem mass spectra (MS/MS) of: (A) a tryptic peptide ion from a peptide mixture affinity-purified with an anti-acetyllysine antibody from tryptic peptides of HeLa nuclear extracts, (B) a peptide mixture containing three synthetic peptides corresponding to the sequences identified using (A), (C) a tryptic peptide ion of histone H4, and (D) a peptide mixture from two synthetic peptides corresponding to the sequences identified using (C);

FIG. 3 shows the MS/MS analysis of individual synthetic peptides: (A) Peptide No. 1, (B) Peptide No. 12, (C) Peptide No. 13, (D) Peptide No. 2, and (E) Peptide No. 3;

FIG. 4 shows the peak assignment of fragment ions found in the spectrum shown in FIG. 2(A) for identifying: (A) lysine propionylated Peptide No. 1, (B) lysine-butyrylated Peptide No. 12, in which the square labels show the fragment ions specific to Peptide No. 12 compared to Peptide No. 1, (C) lysine-butyrylated Peptide No. 13, in which the circle labels show the fragment ions specific to Peptide No. 13 compared to Peptide No. 1;

FIG. 5 shows the peak assignment of fragment ions found in the spectrum shown in FIG. 2(C) for identifying: (A) lysine-propionylated Peptide No. 2, and (B) lysine-propionylated Peptide No. 3, in which the triangle labels show the fragment ions specific to Peptide No. 3 compared to Peptide No. 2;

FIG. 6 shows an autoradiograph of core histone proteins propionylated and butyrylated in vitro with a purified acetyltransferase in the presence of either (¹⁴C)-propionyl-CoA or (¹⁴C)-butyryl-CoA, as indicated;

FIG. 7 shows an autoradiograph of p53 propionylated and butyrylated in vitro with a purified acetyltransferase in the presence of either (¹⁴C)-propionyl-CoA or (¹⁴C)-butyryl-CoA, as indicated; and

FIG. 8 shows an illustration of lysine propionylation and butyrylation sites in histone H4, in which the normal labels show lysine-acetylation and methylation sites identified previously, the circle labels show newly discovered in vivo lysine-modification sites, and the square labels show newly discovered in vitro lysine-modification sites (SEQ ID NO:18).

FIG. 9 shows in vitro lysine propionylation sites in p53 catalyzed by p300 (SEQ ID NO: 19).

FIG. 10 shows in vitro lysine propionylation sites in histone H4 catalyzed by CBP (SEQ ID NO:20).

FIG. 11 shows in vitro lysine propionylation sites in p300 by autopropionylation (SEQ ID NO:21).

FIG. 12 shows in vitro lysine propionylation sites in CBP by autopropionylation (SEQ ID NO:22).

FIG. 13 shows in vitro lysine butyrylation sites in p53 catalyzed by p300 (SEQ ID NO:23).

FIG. 14 shows in vitro lysine butyrylation sites in histone H4 catalyzed by CBP (SEQ ID NO:24).

FIG. 15 shows in vitro lysine butyrylation sites in p300 by autobutyrylation (SEQ ID NO:25).

FIG. 16 shows in vitro lysine butyrylation sites in CBP by autobutyrylation (SEQ ID NO:26).

FIGS. 17A-C. FIG. 17A shows the specificity of anti-K^{Buty} antibody. Four peptide libraries were spotted on nitrocellulose membrane with four dilutions and were used to assay the antibody's specificity. The 14-residue randomized peptide libraries have a fixed residue at 8th position, K^{Ac} in lane 1, K^{Prop} in lane 2, K^{Buty} in lane 3, and K in lane 4. Dot-blot analysis was used to test the specificity of the antibody. To perform the dot-blot analysis, 2 ul of peptide diluted in distilled water to different concentrations was spotted on a strip of nitrocellulose membrane and allowed to air dry. The membrane was then assayed in a manner similar to that used in the immunoblot analysis. FIG. 17B shows Western blotting analysis using anti-K^{Buty} antibody, with competition using the randomized peptide libraries with a fixed K (lane 1) or fixed K^{Buty} (lane 2). Lane 3 and 4 are WB controls using anti-H3 and anti-H4 antibodies, respectively. For antibody competition assay, 200 ng of anti-K^{Buty} antibody was incubated with 1 ug of a peptide library with a fixed non-modified lysine (lane 1) or with a fixed butyryllysine (lane 2), respectively. After incubation for 3 hour at room temperature, the antibody was subjected to immunoblot against core histones derived from Hela cells. Histone H3 and H4 antibodies were used to indicate the positions of H3 or H4 (lane 3 and lane 4). FIG. 17C (Top) shows K^{Buty} of H3 and H4 revealed by anti-K^{Buty} antibody. The core histones were prepared from HeLa cells treated with nothing, or trichostatin A (TSA), or sodium butyrate (NaBu), as indicated. FIG. 17C (Bottom) shows H3 and H4 loading controls. To detect the effect of HDAC inhibitors, Hela cells were treated with 2 uM TSA or 50 mM Sodium butyrate (NaBu). The whole cell lysate was extracted 6 hours after treatment with or without HDAC inhibitors and subjected to immunoblot analysis.

FIG. 18 illustrates a strategy for identifying PTM sites among core histones. K^{Buty} is used as an example.

FIG. 19 is an illustration of novel K^{Prop} and K^{Buty} sites identified in core histone proteins (SEQ ID NOS:27, 28 and 29).

### DETAILED DESCRIPTION OF PREFERRED EMBODIMENTS

The present invention relates generally to mapping of lysine modifications in proteins. In particular, the present invention relates to a method for the identification of lysine propionylation and lysine butyrylation involving the use of protein databases, protein synthesis, and mass spectrometry.

Due to the development of a novel method for detecting the presence of protein modifications, two novel, in vivo lysine modifications in histones, lysine propionylation and butyrylation, have been detected. In vitro labeling and peptide mapping by mass spectrometry confirms that two previously known acetyltransferases, p300 and CBP, can catalyze lysine propionylation and lysine butyrylation in both histones and p53. In addition, p300 and CBP can carry out autopropionylation and autobutyrylation in vitro. Taken together, the results conclusively establish that lysine propionylation and lysine butyrylation are novel post-translational modifications. Given the unique roles of propionyl CoA and butyryl-CoA in energy metabolism and significant structural changes induced by the modifications, the two modifications are likely to have important, but distinct functions in the regulation of biological processes.

Other evidence supports the idea that cells can use other short-chain CoAs, such as propionyl- and butyryl-CoA (which are structurally close to acetyl-CoA), to carry out post-translational modifications at lysine residue. First, like acetyl-CoA, propionyl-CoA and butyryl-CoA are high energy molecules, making it thermodynamically feasible to carry out a reaction with a lysine side chain. Second, propionyl-CoA and butyryl-CoA are structurally similar to acetyl-CoA, with a difference of only one or two CH₂. Third, propionyl-CoA and butyryl-CoA are present at high concentration in cells. In the case of starved mouse liver, the two CoA's concentrations are only 1-3 times less than acetyl-CoA. Finally, it appears, from structural studies on some histone acetyltransferases (“HATs”) (such as Hat1), that the enzyme has ample space within the cofactor binding pocket to accept propionyl-CoA without steric interference. Despite such evidence, the short-chain CoAs with the exception of acetyl-CoA have not been described as a substrate for protein modification.

Acetyl-CoA can arise during the catabolism of sugars, fatty acids and amino acids. Propionyl-CoA derives only from odd-chain fatty acid and amino acid catabolism, while butyryl-CoA is a metabolic intermediate formed during the P-oxidation of fatty acids as well as a substrate for fatty acid elongation. The concentration of the short-chain CoAs fluctuates depending on diet and cellular physiological conditions. If the rate of the modifications depend on the concentration of the short-chain CoAs, directly or indirectly, it is possible that lysine propionylation and butyrylation may regulate cellular metabolic pathways in response to cellular physiology conditions. Such a scenario then opens up the potential for the biochemical intermediates thus produced to lead to tissue-specific and environmentally-responsive regulatory programs.

The novel detection methods have led to the identification and validation of two novel post-translational protein modifications, propionylation and butyrylation at lysine residue, by a proteomics study. The unbiased global screening involves exhaustive peptide identification by nano-HPLC/MS/MS analysis, protein sequence database search, and manual verification. The resulting propionylated and butyrylated peptides are verified by MS/MS of their corresponding synthetic peptides. Using in vitro labeling with isotopic propionyl CoA and butyryl CoA as well as mass spectrometry, two acetyltransferases, p300 and CBP, were identified that could perform robust lysine modifications at both histones and p53 in vitro. Further more, p300 and CBP can carry out autopropionylation and autobutyrylation at lysine residues in a similar fashion as autoacetylation. Taken together, these results reveal that lysine propionylation and butyrylation are novel lysine modifications that can be catalyzed by acetyltransferases. Given the unique roles of propionyl CoA and butyryl-CoA in energy metabolism, their distinct structure, and significant structural changes induced by the modifications, it is anticipated that lysine propionylation and butyrylation will have important, but likely distinct functions in the regulation of biological processes.

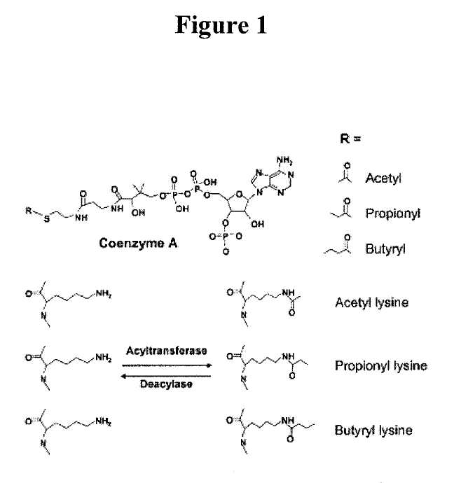

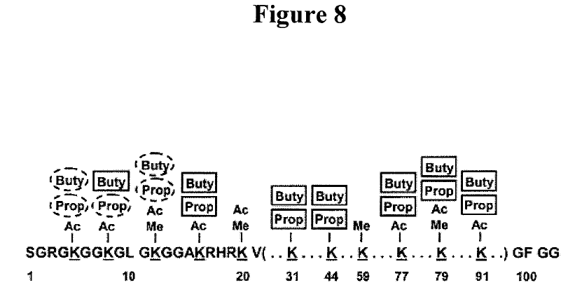

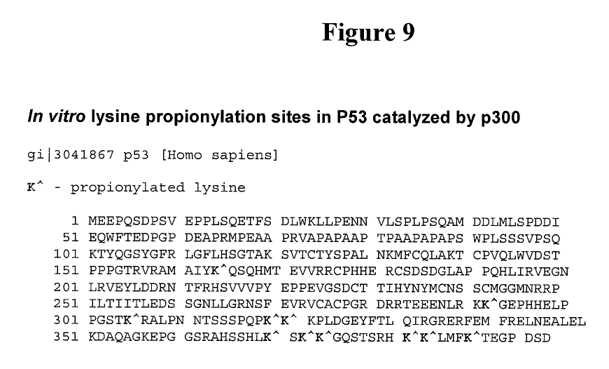

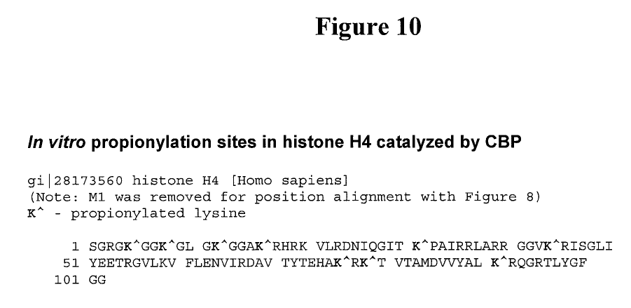

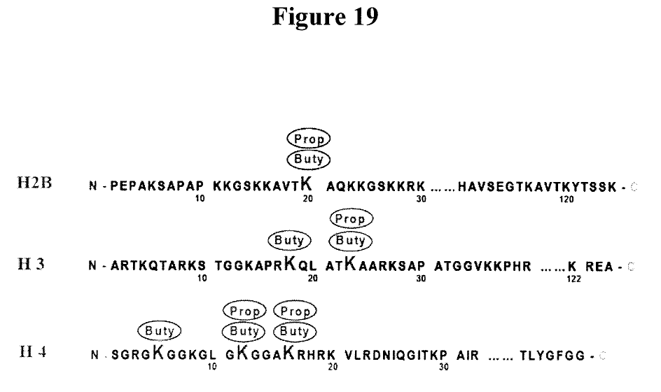

FIG. 1 shows the structures of three short-chain CoAs, acetyl CoA, propionyl CoA, and butyryl CoA, as well as the three modified lysines: acetyllysine, propionyllysine, and butyryllysine. FIG. 8 shows an illustration of new lysine propionylation and butyrylation sites in a histone H4, as detected by the current detection methods. In FIG. 8, the normal labels represent lysine-acetylation and methylation sites identified previously. The circular labels represent new, in vivo lysine-modification sites detected using the current method. The square labels represent new, in vitro lysine-modification sites detected using the current method. FIG. 19 shows an illustration of the new lysine propionylation and butyrylation sites in histones H2B, H3, and H4.

In one embodiment, the current method for identifying and mapping propionylated lysine residues and butyrylated lysine residues in peptides involves a series of steps. First, protein sequence databases, such as NCBl-nr, are searched. These protein sequence databases contain mass spectrometry datasets of peptide spectra. In particular, the databases are searched to locate peptide spectra of peptides that were affinity-purified with anti-acetyllysine antibody. Due to the close similarity between the acetyllysine residue and the propionyllysine residue, propionylated peptides are affinity-purified with the anti-acetyllysine antibody as well. In preferred embodiments, the mass spectrometry datasets of peptide spectra are MS/MS datasets acquired by performing nano-HPLC/LTQ mass spectrometry.

In a next step in the current method, the set of peptide spectra obtained from searching the databases are manually reviewed to identify those known peptides that have propionylated lysine residues and butyrylated lysine residues in addition to the acetylated lysine residues. After identifying those known peptides, synthetic peptides having the same sequence of amino acids can be synthesized. In preferred embodiments, that synthesis is carried out using a protein synthesizer. Free acetylated lysine molecules are used at the positions of the known proteins where acetylated lysine is found. Free protected lysine molecules are used at the positions of the known proteins where propionylated and butyrylated lysine residues are found. These protected lysine molecules have protected side chains. After sequencing, the side chains are removed from the protected lysine residues so that they are no longer protected. Then, propionic acid and butyric acid are used to modify those unprotected lysine residues so that the appropriate modification is established according to the sequence of the known protein. In an even more preferred embodiment, the modification of the lysine residues is carried out with Fmoc chemistry. The free acetylated lysine molecules are Fmoc-Lys(Ac), the free protected lysine molecules are Fmoc-Lys(Mtt), and the side chain protection is methyltrityl (Mtt) side chain protection.

After the synthetic proteins are synthesized, they are analyzed with mass spectrometry. In a preferred embodiment, the synthetic proteins are analyzed by performing an MS/MS analysis using nano-HPLC/LTQ mass spectrometry so that their spectra are similar to those of the known proteins. In another preferred embodiment, the synthetic proteins are first separated on a capillary HPLC column before the mass spectrometry analysis. After the analysis, the spectra of the synthetic proteins are compared to those of the known proteins in order to verify that the identification and mapping of the propionylated lysine residues and butyrylated lysine residues was correct. If the spectra match up, then the identification and mapping was carried out properly.

The ability to identify and detect the presence of propionylated lysine residues and butyrylated lysine residues is invaluable due to the importance of lysine modification to cellular physiology and pathology. Given the widespread applications and huge markets for anti-phosphotyrosine antibody and anti-acetyllysine antibody, the potential for anti-propionyllysine and anti-butyryllysine antibodies as research reagents and reagents for drug screening is vast and promising.

It is also apparent that the propionylation and butyrylation of lysine residues can be catalyzed by known acetyltransferases, such as CBP, p300, Tip60, MOF, and PCAF. In particular, the acetyltransferases CBP and p300 can transfer propionyl CoA or butyryl CoA to lysine residues in proteins in vitro. In preferred embodiments, these catalyzed reactions are carried out on core histones such as H4 and on the protein p53.

### I Antibodies

The present invention provides antibodies that specifically bind to a propionylated lysine or butyrylated lysine or specifically bind to epitopes that contain propionylated lysine or butyrylated lysine. Such antibodies may be made in vivo in suitable laboratory animals or in vitro using recombinant DNA techniques. For example, a polyclonal antibody may be prepared by immunizing an animal with an immunogen comprising propionylated lysine or butyrylated lysine and collecting antisera from that immunized animal. A wide range of animal species can be used for the production of antisera. Typically an animal used for production of anti-antisera is a rabbit, a mouse, a rat, a hamster or a guinea pig.

The amount of immunogen composition used in the production of polyclonal antibodies varies upon the nature of the immunogen, as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. Booster injections also may be given. The process of boosting and titering is repeated until a suitable titer is achieved. When a desired level of immunogenicity is obtained, the immunized animal can be bled and the serum isolated and stored, and/or the animal can be used to generate mAbs (discussed below).

Typically, polyclonal antisera is derived from a variety of different “clones,” i.e., B-cells of different lineage. Monoclonal antibodies (mAbs), by contrast, are defined as coming from antibody-producing cells with a common B-cell ancestor, hence their “mono” clonality. To obtain mAbs, one also initially immunizes an experimental animal, often preferably a mouse, with a propionylated lysine- or butyrylated lysine-containing composition. One would then, after a period of time sufficient to allow antibody generation, obtain a population of spleen or lymph cells from the animal. The spleen or lymph cells can then be fused with cell lines, such as human or mouse myeloma strains, to produce antibody-secreting hybridomas. These hybridomas may be isolated to obtain individual clones which can then be screened for production of antibody to the desired peptide.

Following immunization, spleen cells are removed and fused, using a standard fusion protocol with plasmacytoma cells to produce hybridomas secreting mAbs against the antigen compositions. Hybridomas that produce mAbs to the selected antigens are identified using standard techniques, such as ELISA and Western blot methods. Of importance in identifying antibodies that specifically bind to propionylated lysine or butyrylated lysine is to exclude those antibodies that also bind to unmodified lysine. Hybridoma clones can then be cultured in liquid media and the culture supernatants purified to provide the propionylated lysine- or butyrylated lysine-specific mAbs.

The antibodies of the present invention will find useful application in a variety of procedures, such as ELISA and Western blot methods, as well as other procedures such as immunoprecipitation, immunocytological methods, etc. which may utilize antibodies specific to propionylated lysine or butyrylated lysine. In particular, propionylated lysine- or butyrylated lysine-specific antibodies may be used in assays to detect changes in post-translation modification of proteins.

### II Protein Analysis

The present invention employs methods of separating polypeptides in proteinacious samples. In addition, the present invention employs methods of quantifying and characterizing polypeptides or groups of polypeptides in samples. In particular, the present invention is concerned with determining the post-translational modification (e.g., propionylation and butyrylation) of polypeptides. Methods of separating, quantifying, and characterizing proteins are well known to those of skill in the art and include, but are not limited to, various kinds of chromatography (e.g., anion exchange chromatography, affinity chromatography, sequential extraction, and high performance liquid chromatography) and mass spectrometry.

Mass Spectrometry.

In certain embodiments the methods of the present invention employ mass spectrometry. Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). Mass spectrometry (MS), because of its extreme selectivity and sensitivity, has become a powerful tool for the quantification of a broad range of bioanalytes including pharmaceuticals, metabolites, peptides and proteins.

Of particular interest in the present invention is surface-enhanced laser desorption ionization-time of flight mass spectrometry (SELDI-TOF MS). Whole proteins can be analyzed by SELDI-TOF MS, which is a variant of MALDI-TOF (matrix-assisted desorption ionization-time of flight) mass spectrometry. In SELDI-TOF MS, fractionation based on protein affinity properties is used to reduce sample complexity. For example, hydrophobic, hydrophilic, anion exchange, cation exchange, and immobilized-metal affinity surfaces can be used to fractionate a sample. The proteins that selectively bind to a surface are then irradiated with a laser. The laser desorbs the adherent proteins, causing them to be launched as ions. The “time of flight” of the ion before detection by an electrode is a measure of the mass-to-charge ration (m/z) of the ion. The SELDI-TOF MS approach to protein analysis has been implemented commercially (e.g., Ciphergen).

One- and Two-Dimensional Electrophoresis.

In certain embodiments the present invention employs electrophoresis to separate proteins from a biological sample. Electrophoresis may be performed in one or two dimensions. Typical, one-dimensional gel electrophoresis separates proteins by their molecular mass. Two-dimensional gel electrophoresis is used to generate a two-dimensional array of spots of proteins from a sample. Two-dimensional electrophoresis is a useful technique for separating complex mixtures of molecules, often providing a much higher resolving power than that obtainable in one-dimension separations. Two-dimensional gel electrophoresis can be performed using methods known in the art (See, e.g., U.S. Pat. Nos. 5,534,121 and 6,398,933). Typically, proteins in a sample are separated by, e.g., isoelectric focusing, during which proteins in a sample are separated in a pH gradient until they reach a spot where their net charge is zero (i.e., isoelectric point). This first separation step results in one-dimensional array of proteins. The proteins in one dimensional array is further separated using a technique generally distinct from that used in the first separation step. For example, in the second dimension, proteins separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of the protein.

Proteins in one- or two-dimensional arrays can be detected using any suitable methods known in the art. Staining of proteins can be accomplished with calorimetric dyes (coomassie), silver staining and fluorescent staining (Ruby Red). As is known to one of ordinary skill in the art, proteins can be excised from the gel or transferred to an inert membrane by applying an electric field for further analysis.

Chromatography.

Chromatography is used to separate organic compounds on the basis of their charge, size, shape, and solubilities. A chromatography consists of a mobile phase (solvent and the molecules to be separated) and a stationary phase either of paper (in paper chromatography) or glass beads, called resin, (in column chromatography) through which the mobile phase travels. Molecules travel through the stationary phase at different rates because of their chemistry. Types of chromatography that may be employed in the present invention include, but are not limited to, high performance liquid chromatography (HPLC), ion exchange chromatography (IEC), and reverse phase chromatography (RP). Other kinds of chromatography include: adsorption, partition, affinity, gel filtration and molecular sieve, and many specialized techniques for using them including column, paper, thin-layer and gas chromatography (Freifelder, 1982).

### EXAMPLE 1

### Experimental Procedures

In the examples described below, the following methods and procedures were used.

Synthesis of lysine propionylated and butyrylated peptides. The peptides were synthesized on a Protein Technologies SYMPHONY (Protein Technologies, Inc., Tucson, Ariz.) peptide synthesizer using Fmoc chemistry. All amino acids were purchased from Novabiochem (San Diego, Calif.) and the solvents were obtained from Fisher Science (Fair Lawn, N.J.). Fmoc-Lys(Ac) was used for lysine residues with acetylated side-chains. For lysine residues requiring modification with either butyl or propionyl moieties, an orthogonally protected Fmoc-Lys(Mtt) reagent was used. At the end of the synthesis, prior to removal of the N-terminal Fmoc protecting group, the methyltrityl (Mtt) side-chain protection was removed with 1% trifluoroacetic acid in dichloromethane. The resin was washed 10 times in the acidic solution until the yellow color disappeared. The resin was then treated with 5% diisopropylethylamine to neutralize the trifluoroacetic acid (“TFA”) salt and the free amino group was reacted with either propionic acid or butyric acid, which had been preactivated with HBTU/HOBt. The coupling efficiency was monitored using a quantitative ninhydrin test. After derivatization the resin was treated with 20% piperidine in NMP to remove the Fmoc group and cleaved with 95% TFA, containing thiol scavengers for 90 minutes. The crude peptides were precipitated in diethyl ether and desalted on C-18 RP SEP-PAK (Waters, Milford, Mass.) columns before lyophilization to a dry powder.

In-gel digestion. Protein in-gel digestion, peptide extraction, and peptide cleaning using a μ-C18 Ziptip (Millipore, Billerica, Mass.) were carried out according to traditional methods known in the art (Zhao, et al. 2004).

HPLC/MS/MS Analysis. “HPLC” refers to high performance liquid chromatography. “MS” refers to mass spectrometry. HPLC/MS/MS analysis for mapping propionylation and butyrylation sites in histones, p53, and p300/CBP was carried out in nano-HPLC/LTQ mass spectrometry according to methods already known in the art (Kim, et al. 2006). “LTQ” refers to linear ion trap mass spectrometry. HPLC/MS/MS analysis of tryptic peptides derived from a protein of interest was performed in nano-HPLC/LTQ mass spectrometry. Each tryptic digest was dissolved in 10 μl HPLC buffer A (0.1% formic acid in water (v/v)) and 2 μl were injected into an AGILENT HPLC system (Agilent, Palo Alto, Calif.) using an autosampler. Peptides were separated on a capillary HPLC column, which was prepared having the dimensions: 10 cm length×75 μm ID, 4 μm particle size, 90 Å pore diameter, with JUPITER C12 resin (Phenomenex, St. Torrance, Calif.) and directly electrosprayed into the mass spectrometer using nano-spray source. The LTQ mass spectrometer was operated in the data-dependant mode acquiring fragmentation spectra of the ten strongest ions respectively.

Protein sequence database search and manual verification. All MS/MS spectra were searched against the NCBlnr protein sequence database with the specification of lysine modification using the MASCOT database search engine. All lysine propionylated or butyrylated peptides identified with a MASCOT score greater than 20.0 were manually examined with the rules previously described in Chen, et al. (2005). All lysine propionylation or butyrylation sites were identified by consecutive b- or y-ions so that the possibilities that propionylation (+56 Da) or butyrylation (+70 Da) occurring on adjacent residues were eliminated.

In vitro propionylation and butyrylation assay. In vitro propionylation and butyrylation assays were carried out essentially according to methods known in the art (Gu and Roeder, 1997) with some modifications. The FLAG-p300, CBP-HA, FLAG-MOF, and FLAG-PCAF proteins were purified from the transfected 293 cells and GST-Tip60 and GST-p53 from bacteria to homogeneity under stringent conditions (500 mM NaCi+1% Triton X-100). 10 μl reactions contained 50 mM Tris pH 7.9, 10% glycerol, 1 mMDTT, 10 mM sodium butyrate, 1 μl of (¹⁴C)-acyl-CoA (55 mci/mmol; acetyl-CoA from Amersham, Piscataway, N.J., and propionyl-CoA and butyryl-CoA from ARC, Inc., St. Louis, Mo.). Two and a half μg of substrates (core histones or GST-p53) and about 20 to 100 ng of the enzyme protein, as indicated, and incubated at 30° C. for 1 hour. The reaction mixture was then subject to electrophoresis on SDS-PAGE gels, followed by either autoradiography or Coomassie Blue staining.

Mapping in vitro lysine-propionylation and lysine-butyrylation sites catalyzed by different acetyltransferases. The substrate of interest was incubated with an acetyltransferase (CBP, p300, Tip60, MOF, PCAF) at an enzyme-to-substrate ratio of 1:10 and a CoA. To determine autopropionylation or autobutyrylation sites, only the enzyme of interest was used for the in vitro reaction. The protein mixture was resolved in SDS-PAGE. The protein of interest was excised and in-gel digested with trypsin. The resulting tryptic peptides were analyzed by nano-HPLC/MS/MS in a LTQ mass spectrometer and protein sequence database search for mapping protein modification sites using the procedure described above.

### EXAMPLE 2

### Initial Detection of Lysine Propionylated and Butyrylated Peptides in Histone H4 Protein

To identify lysine-propionylated peptides, the MS/MS datasets of affinity-enriched acetyllysine-containing tryptic peptides acquired in nano-HPLC/LTQ mass spectrometry were searched. The peptides were affinity purified with anti-acetyllysine antibodies. The study on lysine-acetylation proteomics was published previously in Kim et al. (2006). During the protein sequence database search, the lysine was considered as unmodified, acetylated, or propionylated. The database search and manual verification of peptides hits led to the identification of eleven lysine-propionylated histone H4 peptides, shown in Table 1 below. The propionylated lysines are indicated in the table as K̂. The symbol K* designates acetylated lysines.

TABLE 1

| SEQ ID NO. | Protein | | | No. of Propionyl- |
|---|---|---|---|---|
| (peptide No.) | Name | gi# | Sequence | Lys Site |
| 1 | Histone 4, H4 | 28173560 | GK\hat{ }GGK*GLGK\hat{ }GGAK*R | 2 |
| 2 | Histone 4, H4 | 28173560 | GGK\hat{ }GLGK*GGAK*R | 1 |
| 3 | Histone 4, H4 | 28173560 | GGK*GLGK\hat{ }GGAK*R | 1 |
| 4 | Histone 4, H4 | 28173560 | GK\hat{ }GGK*GLGK*GGAK*R | 1 |
| 5 | Histone 4, H4 | 28173560 | GK*GGK\hat{ }GLGK*GGAK*R | 1 |
| 6 | Histone 4, H4 | 28173560 | GK*GGK*GLGK\hat{ }GGAK*R | 1 |
| 7 | Histone 4, H4 | 28173560 | GK\hat{ }GGK*GLGK*GGAK | 1 |
| 8 | Histone 4, H4 | 28173560 | GK*GGK\hat{ }GLGK*GGAK | 1 |
| 9 | Histone 4, H4 | 28173560 | GK*GGK*GLGK\hat{ }GGAK | 1 |
| 10 | Histone 4, H4 | 28173560 | GGK*GLGK\hat{ }GGAK | 1 |
| 11 | Histone 4, H4 | 28173560 | GGK\hat{ }GLGK*GGAK | 1 |

The same datasets were searched again for the lysine butyrylated peptides, in which the lysine was considered unmodified, or acetylated, or butyrylated. The analysis identified two additional histone H4 peptides with lysine butyrylation sites, shown in Table 2 below as K″.

TABLE 2

| SEQ ID NO. | Protein | | | No. of Butyryl- |
|---|---|---|---|---|
| (peptide No.) | Name | gi# | Sequence | Lys Site |
| 12 | Histone 4, H4 | 28173560 | GK“GGK*GLGK*GGAK*R | 1 |
| 13 | Histone 4, H4 | 28173560 | GK*GGK*GLGK“GGAK*R | 1 |

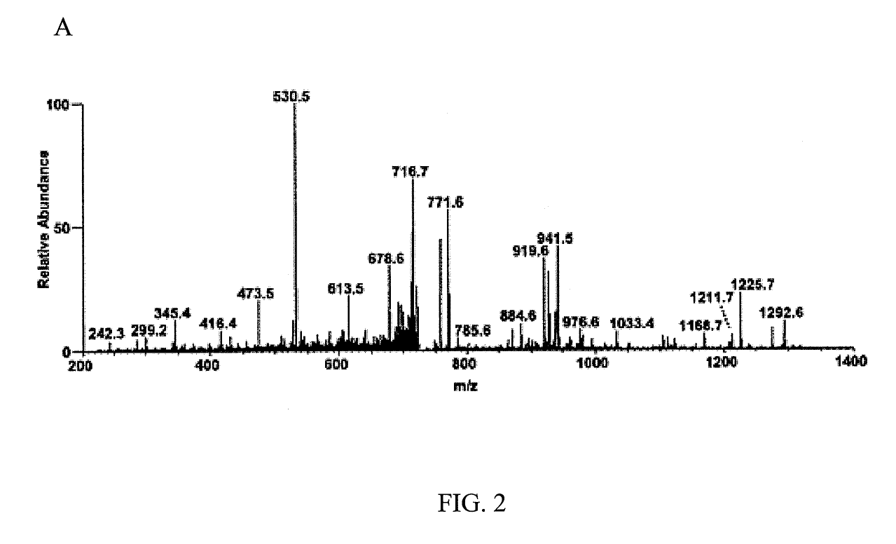

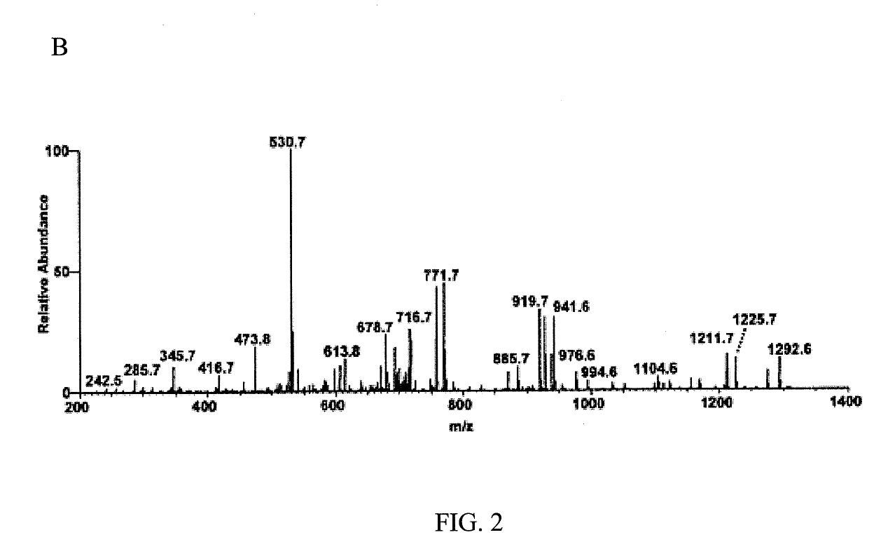

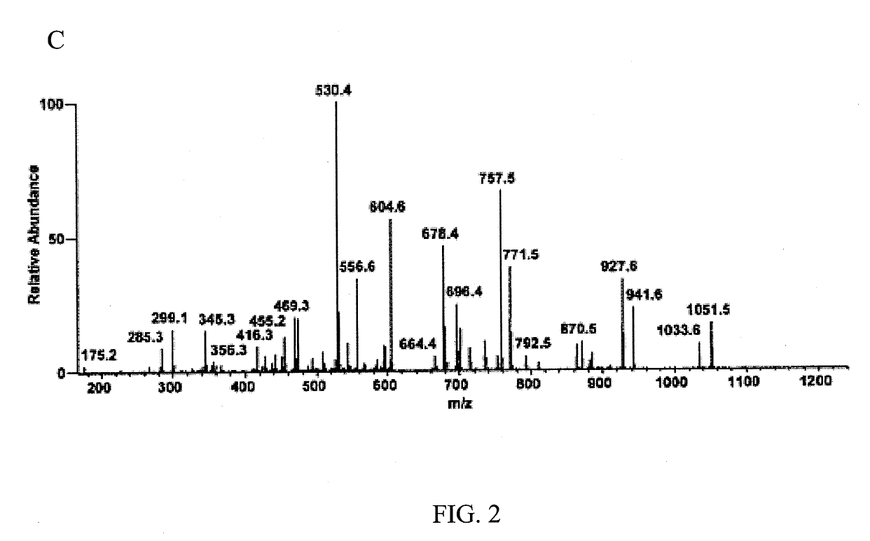

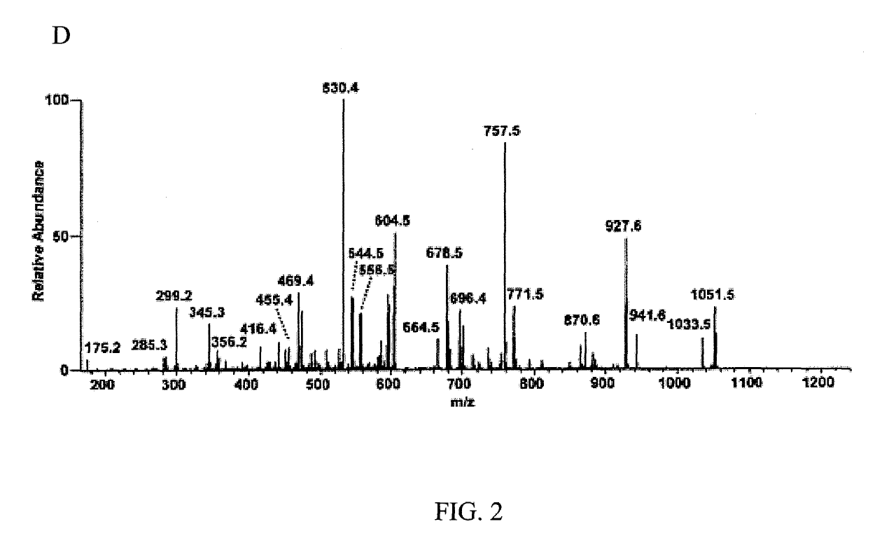

The tandem mass spectrum (MS/MS) of a tryptic peptide ion from a peptide mixture that was affinity-purified with an anti-acetyllysine antibody from tryptic peptides of HeLa nuclear extracts was obtained. FIG. 2 shows the tandem mass spectrum (MS/MS) used to identify the lysine-propionylated and lysine-butyrylated peptides shown in Tables 1 and 2. As examples, the spectrum shown in FIG. 2(A) identified Peptide No. 1 from Table 1 above. The spectrum in FIG. 2(C) identified Peptide No. 2 from Table 1 above. Analysis of the two spectra indicated that the spectra were derived from more than one peptide because: (i) the multiple peak-pairs with mass difference of 14 Da were observed in both spectra; (ii) the peptides had the same molecular weights; and (iii) the peptides were co-eluted.

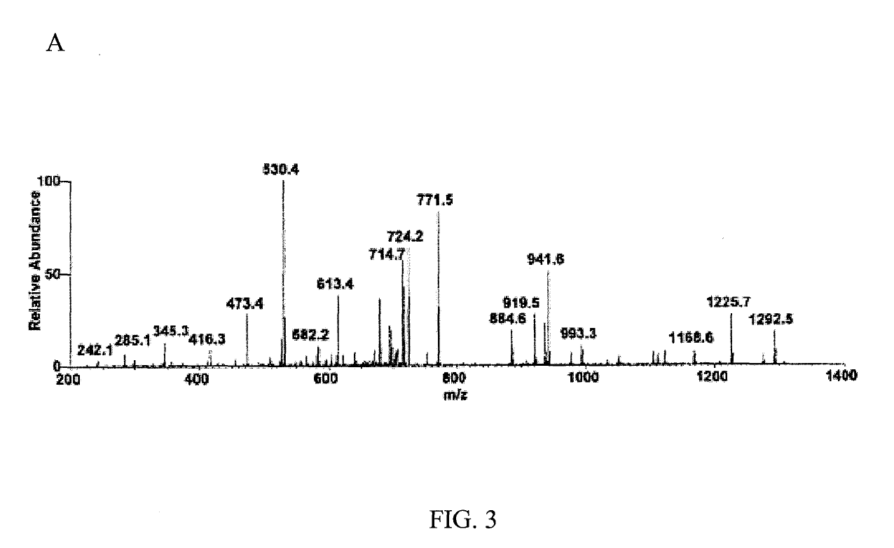

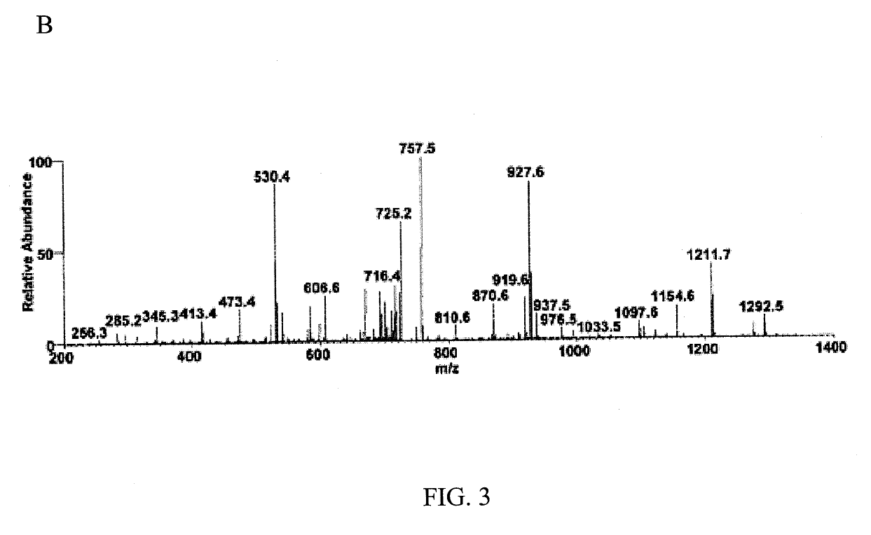

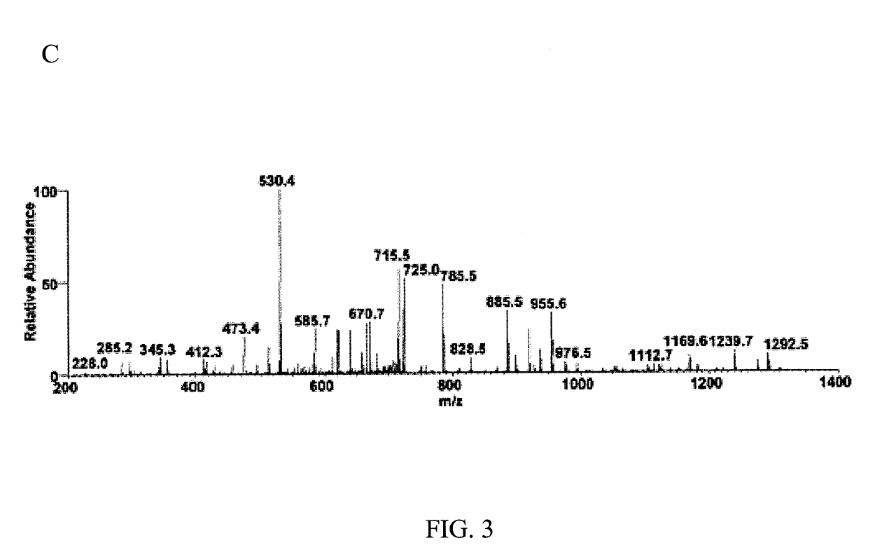

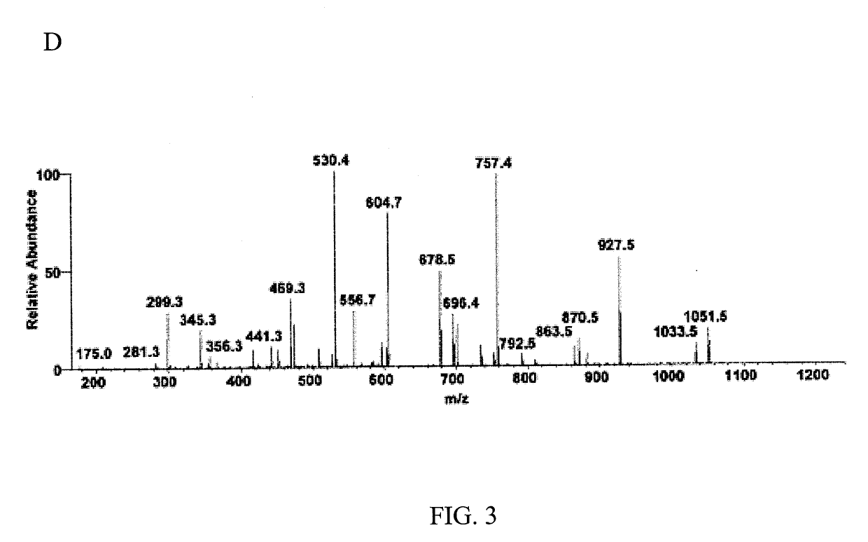

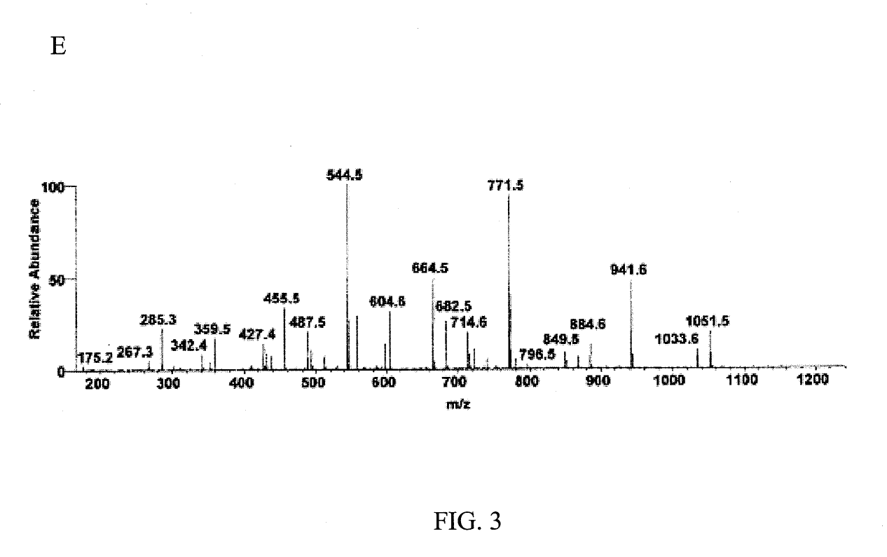

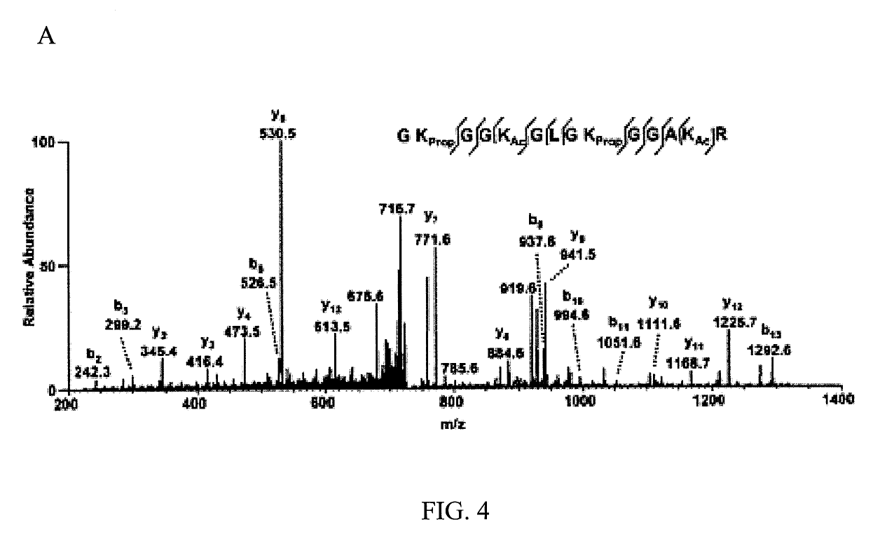

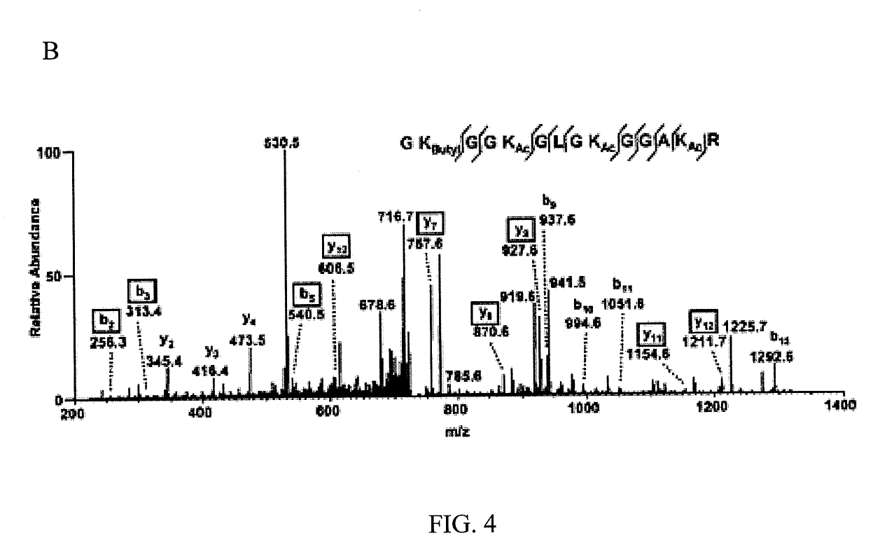

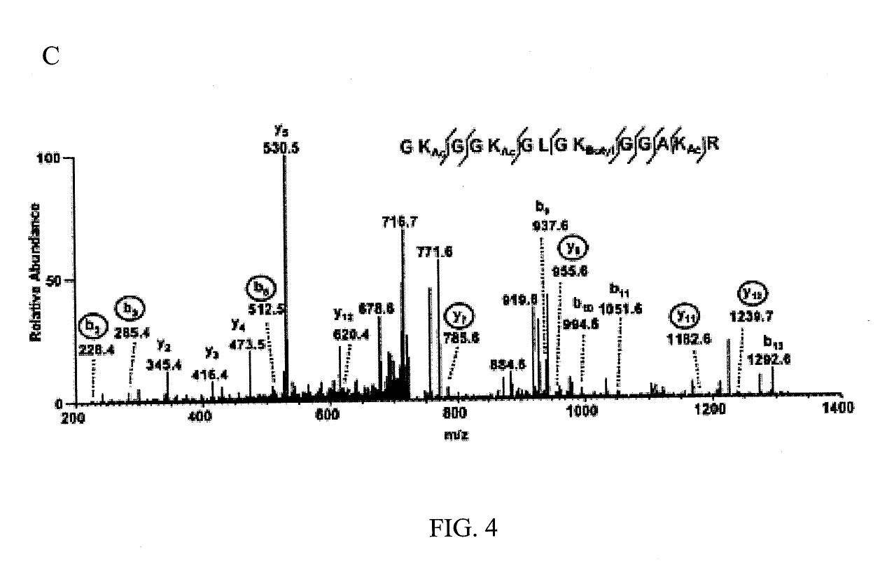

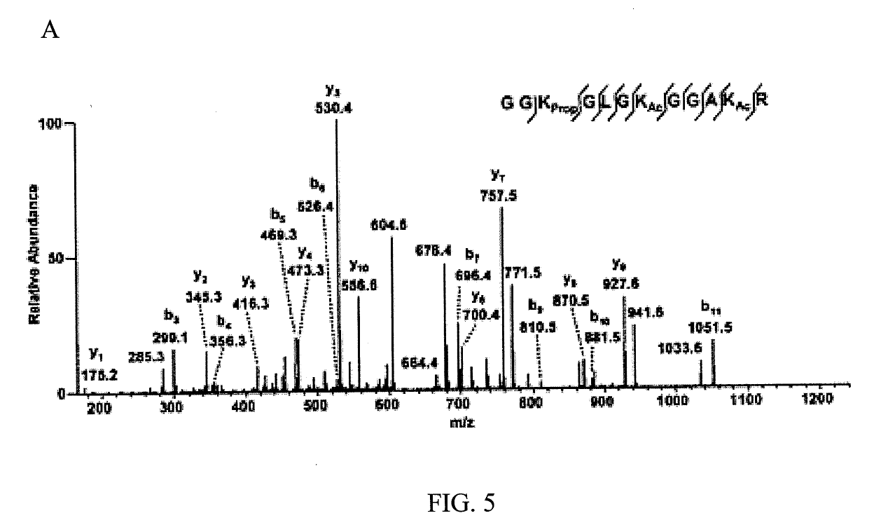

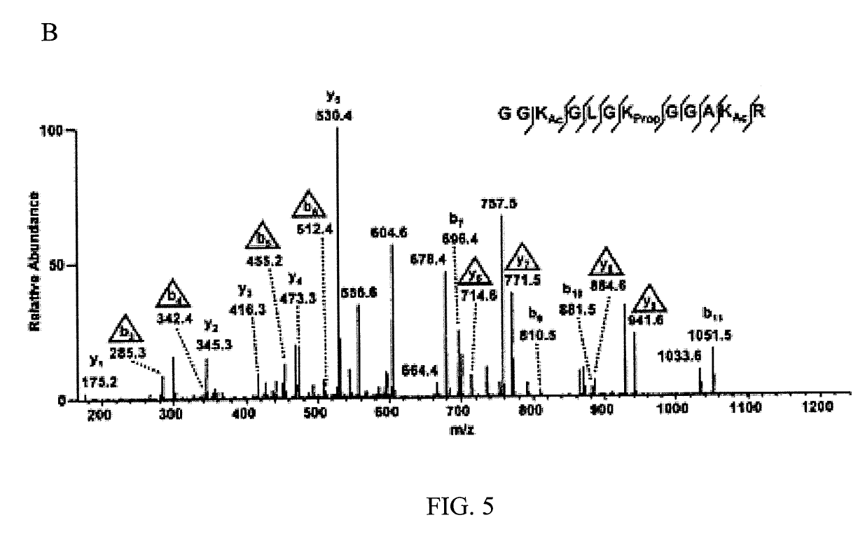

The fragmentation spectrum of synthetic Peptides Nos. 1, 12, 13, 2, and 3 are shown in FIG. 3. The peak assignments in the spectrum of FIG. 2(A) for the identification of the lysine-propionylated peptide and the two lysine-butyrylated peptides are shown in FIG. 4. The square labels show the fragment ions specific to Peptide No. 12 compared to Peptide No. 1. The circle labels show the fragment ions specific to Peptide No. 13 compared to Peptide No. 1. The peak assignments in the spectrum of FIG. 2(C) for the identification of two lysine-propionylated peptides are shown in FIG. 5. The triangle labels show the fragment ions specific to Peptide No. 3 compared to Peptide No. 2. Thus, the remaining peaks in the spectrum shown in FIG. 2(A) were explained by the two additional lysine-butyrylated peptides, Peptides No. 12 and No. 13 shown in Table 2 above. These spectra are shown in particular in FIGS. 3(B), 3(C), 4(A), 4(B), and 4(C). Likewise, an additional peptide isomer, Peptide No. 3 from Table 1 above, was identified in the spectrum of FIG. 2(C), as shown in FIGS. 3(E), 5(A), and 5(B).

The chemical nature of an identified peptide can be confirmed by MS/MS of their corresponding synthetic peptides, a gold standard for verification of peptide identification and chemical identity. To ascertain identification of the propionylated and butyrylated peptides, MS/MS of 3 synthetic peptides (identified from the spectrum in FIG. 2(A)) were analyzed as shown in FIGS. 3(A)-(C). A mixture of the synthetic peptides corresponding to Peptides Nos. 1, 12, and 13 with a ratio of 4:2:1 matched perfectly with the spectrum in FIG. 2(A), verifying the identification of the three peptides (FIG. 2(B)). Likewise, Peptides 2 and 3 were confirmed by MS/MS of the two peptides with a ratio of 2:1.

Lysine propionylation was identified at K5, K8, and K12, as well as lysine butyrylation at K5 and K12 of histone H4 (as shown in Tables 1 and 2). The K5, K8, and K12 of histone H4 is known to be acetylated, while the K12 is the subject of lysine methylation. Lysine acetylation at the four H4 lysine residues is associated with transcriptional activation, transcriptional silencing, chromatin high-order structure, and DNA repair (Peterson et al., 2004 and Shia et al., 2006). Some of the acetyllysine residues (e.g., K8 of histone H4) provide a docking site to recruit a bromodomain-containing chromatin remodeling enzyme SWI/SNF. While biological functions of lysine propionylation and butyrylation in histones remain unknown, and without being bound by theory, it is possible that propionyllysine or butyryllysine are involved in the interaction or recruiting of a distinct set of proteins or enzymes to control chromatin's structure and transcriptional activities.

### EXAMPLE 3

### Propionylation and Butyrylation of Core Histones Catalyzed by P300/CBP

Because the histone H4 can be propionylated and butyrylated in vivo, it was next tested if core histones could be propionylated and butyrylated in vitro by acetyltransferases, using either ¹⁴C-propionyl CoA or ¹⁴C-butyryl CoA. Five acetyltransferases were tested, CBP, p300, Tip60, MOF and PCAF. CBP and p300 are known acetyltransferases for K5, K8, K12, and K16 of histone H4.

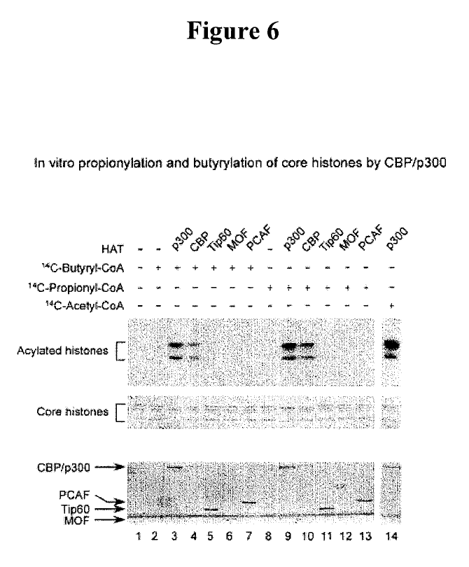

The core histones were incubated with the purified acetyltransferase in the presence of either (¹⁴C)-propionyl-CoA or (¹⁴C)-butyryl-CoA. The protein mixtures were then resolved in SDS-PAGE and visualized by autoradiography. The levels of the core histone substrates and the acetyltransferases were visualized by Coomassie Blue staining. CBP and p300 showed significant activities to catalyze both modifications in histone H3 and H4, as shown in FIG. 6. On the other hand, no significant propionylation and butyrylation products were detected for the other three acetyltransferases, Tip60, MOF, and PCAF.

To corroborate in vitro modification reaction at lysine residues, nano-HPLC/mass spectrometric analysis was used to map the CBP-catalyzed, lysine-modified residues in histone H4. K5, K8, K12, K16, K31, K44, K77, K79 and K91 were found to be both propionylated and butyrylated by CBP. Together, these data establish that histone H3 and H4 can be lysine propionylated and butyrylated directly by CBP and p300 in vitro.

### EXAMPLE 4

### In Vitro Propionylation and Butyrylation of P53 Catalyzed by P300/CBP

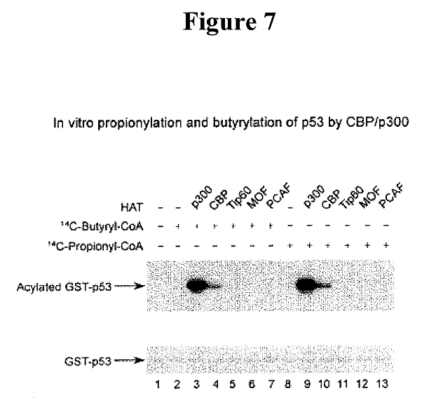

To examine if acetyltransferases can catalyze lysine propionylation and butyrylation reactions in non-histone proteins, in vitro propionylation and butyrylation reactions in p53 were evaluated. CBP/p300 is a co-activator of p53 that affects its transcriptional activity and modulates its biological functions (Gu et al., 1997 and Avantaggiati et al., 1997). Multiple lysine residues in p53, including K120, K320, K305, K370, K372, K373, K381, and K382, can be acetylated, of which the last five lysine residues were known to be modified by CBP/p300 (Gu et al., 1997, Tang et al., 2006, and Sakaguchi et al., 1998). Given the fact that CBP/p300 are the acetyltransferases for p53 and that they have enzymatic activities for lysine propionylation and butyrylation in histones, it was tested if the HATs could catalyze similar reactions in p53. Toward this aim, the in vitro enzymatic reactions as described above for p53 were repeated. Again, only two of the five acetyltransferases, CBP and p300, could carry out propionylation and butyrylation reaction at p53 at a significant reaction rate under the experimental conditions, as shown in FIG. 7. Interestingly, p300 shows higher catalytic activity than CBP for p53. In contrast, the two enzymes have comparable activities in histones.

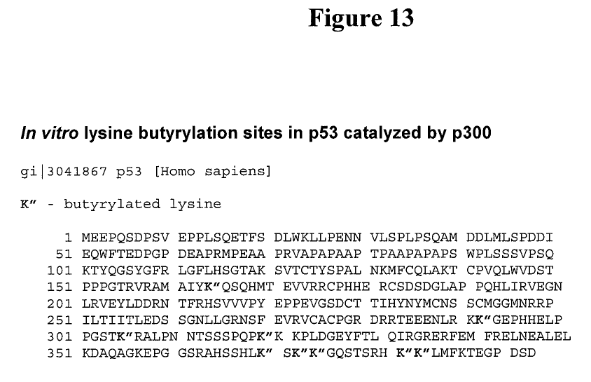

To establish the specificity of propionylation and butyrylation at lysine residues, we again used mass spectrometry to analyze the propionylation and butyrylation sites at p53, after in vitro enzymatic reaction with an appropriate CoA and p300. The analysis led to the identification of eleven lysine propionylation sites on K164, K292, K305, K319, K320, K370, K372, K373, K381, K382 and K386, and nine lysine butyrylation sites on K164, K292, K305, K319, K370, K372, K373, K381 and K382. See FIGS. 9 and 13.

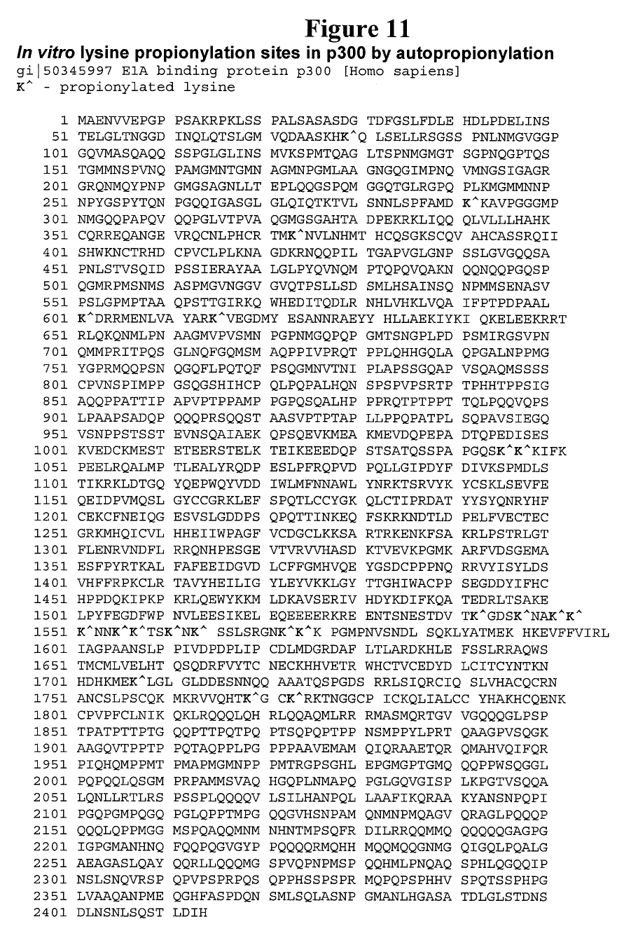

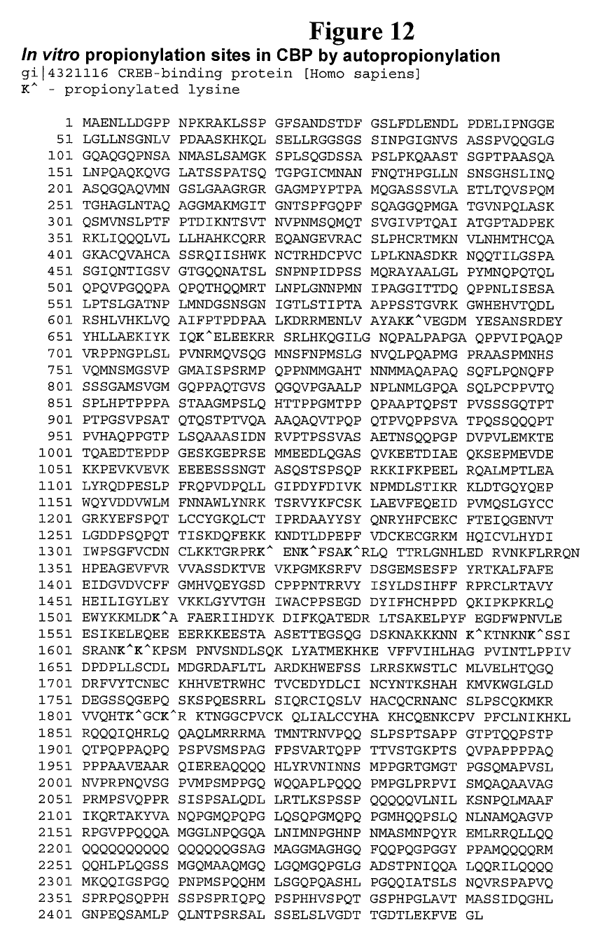

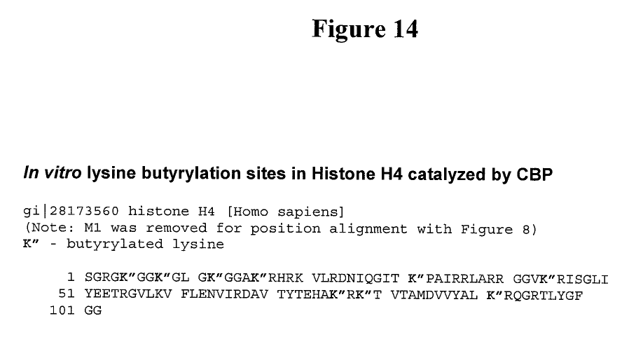

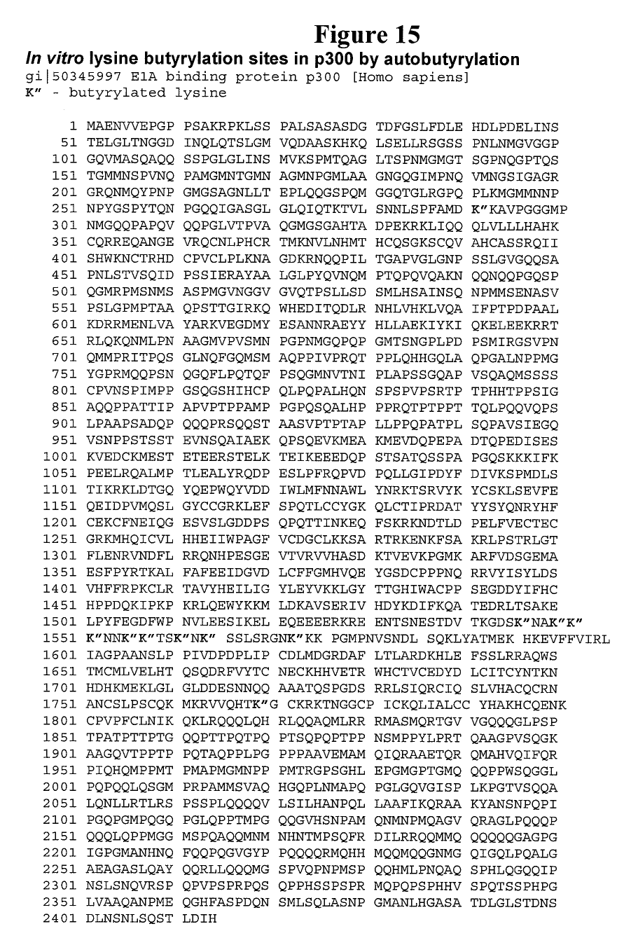

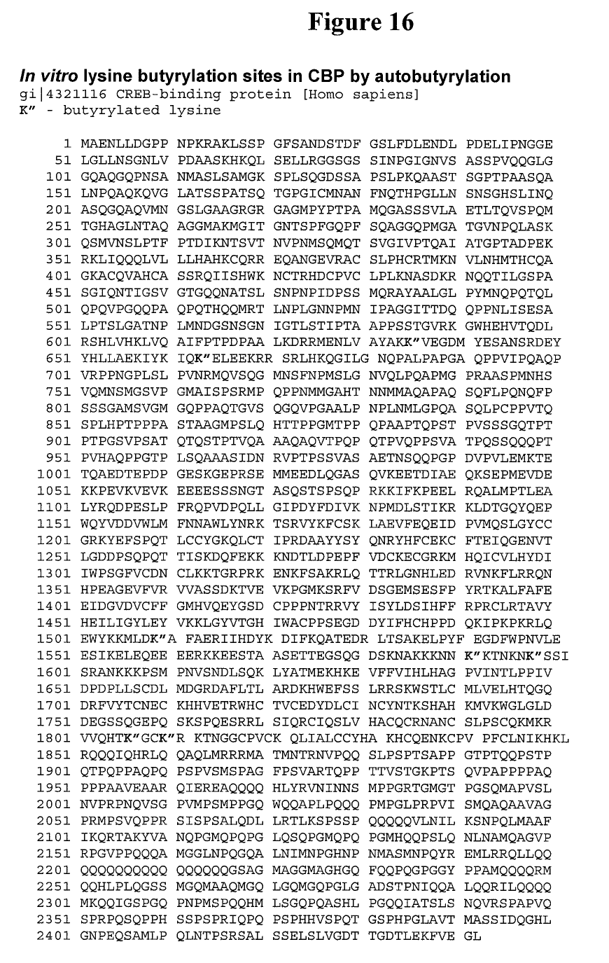

CBP and p300 are acetyltransferases that can catalyze autoacetylation reactions. To test if the proteins could carry out autopropionylation and autobutyrylation reactions, the modification sites at p300 and CBP were mapped. Twenty-one lysine-propionylation sites and eleven lysine-butyrylation sites were localized in p300, while twelve lysine-propionylation sites and seven lysine-butyrylation sites were mapped in CBP. See FIGS. 11, 12, 15, and 16, and Table 3 below. Identification of propionylated and butyrylated peptides in non-histone proteins, p53 and p300, suggests the possibility that the two modifications are not restricted in histones.

TABLE 3

| In vitro analysis | p53 | p300 | Histone H4 | CBP |
|---|---|---|---|---|
| Propionyl-Lys | 11 | 21 | 9 | 12 |
| Butyryl-Lys | 9 | 11 | 9 | 7 |

### EXAMPLE 5

### Antibodies Specific for Propionylated and Butyrylated Peptides

Generation of peptide libraries for antibody generation. Pan-antibodies were generated using the strategy described by Zhang et al. (2002). Briefly, the following degenerate peptide libraries containing a fixed modified lysine surrounded on each side by six random amino acids were synthesized: CXXXXXXKXXXXXX (SEQ ID NO:14), CXXXXXXK^{Prop}XXXXXX (SEQ ID NO:15), CXXXXXXK^{Ac}XXXXXX (SEQ ID NO:16, and CXXXXXXK^{Buty}XXXXXX (SEQ ID NO:17), where X is a mixture of 19 amino acids, excluding cysteine. The peptide libraries were synthesized by solid phase peptide synthesis using conventional Fmoc chemistry and derivatized Fmoc-K^{Ac}, Fmoc-K^{Prop} and Fmoc-K^{Buty} residues. Synthesis was controlled such that each of the 19 amino acid residues would be incorporated at similar frequencies at each position.

Generation and purification of pan-specific antibodies. The K^{Buty} peptide library was conjugated to keyhole limpet hemocyanin (KLH), and the resulting conjugate was used to immunize five rabbits (Strategic Biosolutions Inc. (Newark, Del.)). Antibodies cross-reacting to K^{Buty} were purified using the following purification scheme as previously described by Qiang et al. (2005): (i) IgG from the serum was purified over protein A-Sepharose beads; (ii) the purified IgG was then passed over a column containing K^{Buty}-conjugated agarose beads. The K^{Buty}-conjugated beads were synthesized by a one-step reaction between commercial lysine-conjugated agarose beads and p butyric anhydride under pyridine/THF (1:10, v/v) overnight. Complete acylation at the lysine side chain was confirmed by the conventional ninhydrin test that detects free amino groups.

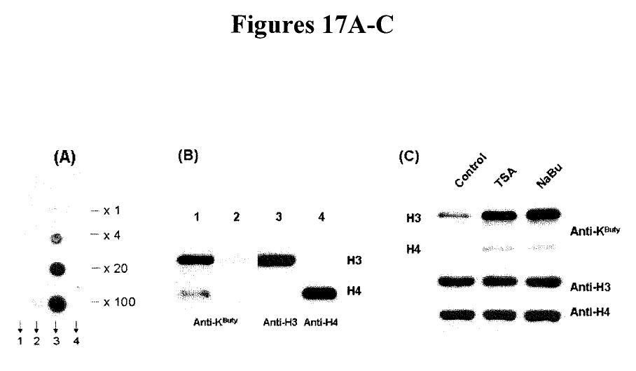

The specificities of the K^{Buty}-specific pan antibodies were evaluated using four peptide libraries: CXXXXXXKXXXXXX (SEQ ID NO:14), CXXXXXXK^{Ac}XXXXXX (SEQ ID NO:16), CXXXXXXK^{Prop}XXXXXX (SEQ ID NO:15), and CXXXXXXK^{Buty}XXXXXX (SEQ ID NO:17). The dot-spot assay with multiple dilutions was used for the analysis (FIG. 17A). The assay showed that the pan-specific K^{Buty} antibody had more than 20-40-fold greater affinity for K^{Buty} than for the other two post-translational modifications (PTMs). Thus, the antibodies have sufficient specificity for detection of their respective PTMs.

Detection of K^{Buty} in histones. To confirm the presence of K^{Buty} in histones, Western blotting analysis was performed using the pan-specific K^{Buty} antibody. Briefly, core histone preparations from HeLa cells were resolved by SDS-PAGE and analyzed by Western blotting using the antibodies, with or without competition from the corresponding peptide library (CXXXXXXK^{Buty}XXXXXX (SEQ ID NO:17)). Strong signals for K^{Buty} were detected in both histones H3 and H4 (FIG. 17B) that could be efficiently competed out by the CXXXXXXK^{Buty}XXXXXX (SEQ ID NO:17) peptide library, suggesting that K^{Buty} residues were present in both H3 and H4 and that the antibody was specific.

It was also demonstrated that the K^{Buty} level was dramatically induced by sodium butyrate (20 mM for 6 hours) and trichostatin A (10 uM, 6 Hours), a class I and class II HDAC inhibitor (FIG. 17C). This data indicates that K^{Buty} specific antibodies can be used to detect the changes in the butyrylation of lysine residues in histones.

Identification of K^{Prop} and K^{Buty} sites in histones from HeLa cells. Initial studies identified K^{Prop} at K5, K8, and K12, and K^{Buty} at K5 and K12 in histone H4. This initial identification used MS/MS data from peptides affinity-purified from a tryptic digest of HeLa nuclear extract using an anti-K^{Ac} antibody (Kim et al., 2006). Since all the identified peptides contained K^{Ac} (Chen et al., 2007) in combination with either K^{Prop} or K^{Buty}, it is believed that the peptides were isolated because of the affinity of the antibody for K^{Ac} residues. Accordingly, this analysis is unlikely to identify K^{Prop}- and K^{Buty}-containing peptides of low abundance or that lack a K^{Ac}.

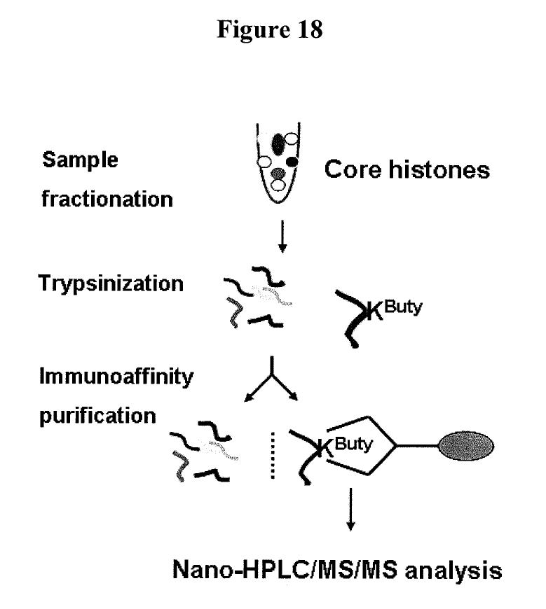

To identify other possible K^{Prop} and K^{Buty} sites in histones, a proteomics screening was performed using a strategy described previously for lysine acetylation (FIG. 18) (Kim et al., 2006). Briefly, core histones from HeLa cells were digested with trypsin and the tryptic peptides were subjected to affinity purification using either anti-K^{Prop} or anti-K^{Buty} antibodies. Bound peptides were eluted and analyzed by nano-HPLC mass spectrometry in an LTQ mass spectrometer. With the MASCOT algorithm, the resulting MS/MS data were used to search the NCBI protein sequence database to i) identify the peptides, and ii) map propionylation and butyrylation sites, allowing lysine residues to be unmodified, K^{Ac}, K^{Prop}, or K^{Buty}. All identified peptides were manually verified using a procedure previously described (Chen et al., 2005). The study identified 2 K^{Prop} sites in histone H3, 1 K^{Prop} site in H2B, and 4 K^{Prop} sites in H4, as well as 1 K^{Buty} site in H2B, 4 K^{Buty} sites in H3, and 3 K^{Buty} sites in H4 (FIG. 19).

### REFERENCES CITED

The following references, to the extent that they provide exemplary procedural or other details supplementary to those set forth herein, are specifically incorporated herein by reference.
U.S. Pat. No. 5,534,121
U.S. Pat. No. 6,398,933

Avantaggiati et al., Cell, 89: 1175-1184, 1997.
Boyne et al., J. Proteome Res., 5:248-253, 2006.
Chen et al., J. Proteome Res., 4:998-1005, 2005.
Chen et al., Mol. Cell Proteomics, 6:812-819, 2007.
Garcia et al., J. Biol. Chem., 282(10):7641-7655, 2007.
Gu and Roeder, Cell, 90:595-606, 1997.
Gu et al., Nature, 387:819-823, 1997.
Jenuwein and Allis, Science, 293:1074-1080, 2001.
Kim et al., Mol. Cell, 23:607-618, 2006.
Medzihradszky et al., Mol. Cell Proteomics, 3:872-886, 2004.
Peterson and Laniel, Curr. Biol., 14:R546-551, 2004.
Qiang et al., J. Immunoassay Immunochem., 26:13-23, 2005.
Sakaguchi et al., Genes Dev., 12: 2831-2841, 1998.
Shia et al., Genome Biol., 7:217, 2006.
Tang et al., Mol. Cell, 24:827-839, 2006.
Zhang et al., J. Biol. Chem., 277:39379-39387, 2002.
Zhao et al. Anal. Chem., 76:1817-1823, 2004.

What is claimed is:

1. A method for detecting a propionyllysine or butyryllysine in a polypeptide comprising:
(a) obtaining a sample comprising polypeptides;
(b) separating the polypeptides by molecular weight;
(c) contacting one or more of the separated polypeptides with an antibody that specifically binds with a polypeptide having a propionyllysine or butyryllysine, but does not substantially bind with a polypeptide that does not have a propionyllysine or butyryllysine; and
(d) detecting the binding of the antibody to the polypeptides, whereby antibody binding to the polypeptides indicates the presence of the propionyllysine or butyryllysine in the polypeptides.

2. The method of claim 1, further comprising immobilizing the polypeptides on a solid support prior to contacting the polypeptides with the antibody.

3. The method of claim 1, further comprising immobilizing the antibody on a solid support prior to contacting the polypeptides with the antibody.

4. The method of claim 1, wherein in the antibody specifically binds the propionyllysine in the polypeptides.

5. The method of claim 1, wherein in the antibody specifically binds the butyryllysine in the polypeptides.

6. The method of claim 1, wherein the antibody's ability to specifically bind to the propionyllysine or butyryllysine is independent of amino acid sequences adjacent to the propionyllysine or butyryllysine.

7. The method of claim 1, wherein the antibody's ability to specifically bind to the propionyllysine or butyryllysine is dependent on amino acid sequences adjacent to the propionyllysine or butyryllysine.

8. The method of claim 1, wherein the detecting of the binding of the antibody to the polypeptides comprises Western blotting.

9. The method of claim 1, wherein separating the polypeptides comprises heating the sample to a temperature sufficient to denature the polypeptides in the sample without significantly degrading peptide bonds of the polypeptides.

10. The method of claim 9, wherein the sample is treated with an enzyme inhibitor during sample preparation.

11. The method of claim 10, wherein the enzyme inhibitor is aprotinin (Trasylol™), phenylmethylsulfonyl fluoride (PMSF), benzamidine, diisopropylfluorophosphate (DIFP), leupeptin, pepstatin, EDTA, EGTA, sodium butyrate, trichostatin A, suberoylanilide hydroxamic acid (SAHA), FK288, nicotinamide, or sirtinol.

12. The method of claim 1, further comprising comparing the amount of propionyllysine and/or butyryllysine modification in at least one of the polypeptides detected in step (d) with the amount of propionyllysine and/or butyryllysine modification in a corresponding polypeptide in a reference sample.

13. The method of claim 12, wherein the sample is obtained from a tissue biopsy or a clinical fluid and the reference sample corresponds is obtained from a corresponding tissue biopsy or a clinical fluid in a diseased organism.

14. The method of claim 1, further comprising comparing the presence or absence of propionyllysine and/or butyryllysine modification in at least one of the polypeptides detected in step (d) with the presence or absence of propionyllysine and/or butyryllysine modification in a corresponding polypeptide in a reference sample.

15. The method of claim 14, further comprising comparing protein activation in the sample with the protein activation in the reference sample.

16. The method of claim 14, wherein the sample is obtained from a tissue biopsy or a clinical fluid and the reference sample corresponds is obtained from a corresponding tissue biopsy or a clinical fluid in a diseased organism.

17. The method of claim 16, further comprising identifying propionyllysine and/or butyryllysine modifications in polypeptides of the sample that are not present in corresponding polypeptides in the reference sample.

18. The method of claim 16, wherein the diseased organism has cancer.

19. The method of claim 1, wherein the sample is a digested biological sample.

20. The method of claim 19, wherein the digested biological sample is a digested crude cell extract, a digested tissue sample, a digested serum sample, a digested urine sample, a digested synovial fluid sample, or a digested spinal fluid sample.

21. The method of claim 12, wherein the sample is treated or is obtained from an organism that was treated with at least one test compound and the reference sample is untreated and is obtained from an untreated organism.

22. The method of claim 21, wherein the test compound is a cancer therapeutic.

23. A method for isolating a group of propionylated or butyrylated peptides from a complex mixture of peptides, comprising:
(a) digesting a proteinaceous material with a proteolytic enzyme or chemical cleavage agent to obtain digested proteinaceous material;
(b) contacting the digested proteinaceous material with an immobilized propionyllysine-specific or butyryllysine-specific antibody; and
(d) isolating from the digested proteinaceous material the target group of propionylated or butyrylated peptides specifically bound by the immobilized propionyllysine-specific or butyryllysine-specific antibody.

24. The method of claim 23, wherein the antibody is an butyryllysine-specific antibody or an antibody that specifically binds to a polypeptide sequence containing butyryllysine.

25. The method of claim 23, wherein the antibody is propionyllysine-specific antibody or an antibody that specifically binds to a polypeptide sequence containing propionyllysine.

26. The method of claim 23, further comprising characterizing the isolated target group of propionylated or butyrylated peptides by mass spectrometry (MS), tandem mass spectrometry (MS/MS), and/or MS3 analysis.

27. The method of claim 26, wherein the mass spectrometry comprises matrix-assisted laser desorption time-of-flight (MALDI-TOF) MS.

28. The method of claim 26, wherein the tandem mass spectrometry comprises liquid chromatography (LC)-MS/MS.

29. The method of claim 26, wherein the MS3 analysis comprises LC-MS3.

30. The method of claim 23, wherein the antibody is immobilized in a chromatography resin within a column.

31. The method of claim 30, wherein the column is coupled to a mass spectrometer.

32. The method of claim 23, further comprising quantifying at least one of the isolated propionylated or butyrylated peptides.

33. The method of claim 32, wherein quantifying the propionylated or butyrylated peptides comprises using stable isotope labeling by amino acids in cell culture (SILAC), isotope-coded affinity tag (ICAT), iTRAQ™, and/or absolute quantification of peptides (AQUA) techniques.

34. The method of claim 23, further comprising comparing the propionyllysine and/or butyryllysine modifications of at least one of the propionylated or butyrylated peptides with the propionyllysine and/or butyryllysine modifications of a corresponding peptide in a reference sample.

35. The method of claim 34, wherein the proteinacious material is obtained from a diseased organism and the reference sample is obtained from a normal organism.

36. The method of claim 34, wherein the proteinacious material is obtained from a tissue biopsy or a clinical fluid and the reference sample is obtained from a corresponding tissue sample or clinical fluid from a diseased organism.

37. The method of claim 34, wherein the proteinacious material is treated or is obtained from an organism that was treated with at least one test compound and the reference sample is obtained from an untreated proteinacious material and untreated organism.

38. The method of claim 23, wherein the proteolytic enzyme is immobilized.

39. The method of claim 23, wherein the digested proteinacious material is treated with a proteolysis inhibitor prior to contacting the digested proteinaceous material with the immobilized propionyllysine-specific or butyryllysine-specific antibody.

40. The method of claim 23, wherein the immobilized antibody is covalently linked to a chromatography resin or noncovalently linked to protein-A- or protein-G-agarose.

41. The method a claim 40, wherein said resin is contained within a column or micropipette tip.

42. An isolated antibody that specifically binds to a propionylated lysine or butyrylated lysine and does not substantially bind to acetylated lysine and unmodified lysine.

43. The isolated antibody of claim 42, wherein the isolated antibody specifically binds to propionylated lysine or a polypeptide sequence containing propionylated lysine.

44. The isolated antibody of claim 42, wherein the isolated antibody specifically binds to butyrylated lysine or a polypeptide sequence containing butyrylated lysine.

45. The isolated antibody of claim 42, wherein the isolated antibody specifically binds to both propionylated lysine and butyrylated lysine or specifically binds to a polypeptide containing both propionylated lysine and butyrylated lysine.

46. The isolated antibody of claim 42, wherein the isolated antibody specifically binds to a propionylated lysine or butyrylated lysine in a histone H2B, H3, or H4 protein.

47. The isolated antibody of claim 46, wherein the isolated antibody specifically binds to a propionylated lysine or butyrylated lysine at histone H2B lysine 20.

48. The isolated antibody of claim 46, wherein the isolated antibody specifically binds to a propionylated lysine at histone H3 lysine 14 or lysine 23.

49. The isolated antibody of claim 46, wherein the isolated antibody specifically binds to a butyrylated lysine at histone H3 lysine 9, lysine 14, lysine 18, or lysine 23.

50. The isolated antibody of claim 46, wherein the isolated antibody specifically binds to a propionylated lysine at histone H4 lysine 5, lysine 8, lysine 12, lysine 16, lysine 31, lysine 44, lysine 77, lysine 79, or lysine 91.

51. The isolated antibody of claim 46, wherein the isolated antibody specifically binds to a butyrylated lysine at histone H4 lysine 5, lysine 8, lysine 12, lysine 16, lysine 31, lysine 44, lysine 77, lysine 79, or lysine 91.

52. The isolated antibody of claim 42, wherein the isolated antibody specifically binds to a propionylated lysine or butyrylated lysine in a p53 protein.

53. The isolated antibody of claim 42, wherein the isolated antibody specifically binds to a propionylated lysine or butyrylated lysine in a p300 protein.

54. The isolated antibody of claim 42, wherein the isolated antibody specifically binds to a propionylated lysine or butyrylated lysine in a CREB-binding protein.

55. A method for in vitro propionylation or butyrylation of at least one lysine residue in a polypeptide comprising incubating a polypeptide with a purified acetyltransferase enzyme, and a propionyl-CoA, a butyryl-CoA, or both a propionyl-CoA and a butyryl-CoA, wherein at least one lysine residue in the polypeptide is propionylated or butyrylated.

56. The method of claim 55, wherein the polypeptide is a core histone.

57. The method of claim 55, wherein the polypeptide is p53.

58. The method of claim 55, wherein the purified acetyltransferase enzyme is CBP or p300.


SEQUENCE LISTING (29 sequences)

SEQ ID NO: 1 - protein, 14 aa, Homo sapiens
GKGGKGLGKGGAKR

SEQ ID NO: 2 - protein, 12 aa, Homo sapiens
GGKGLGKGGAKR

SEQ ID NO: 3 - protein, 12 aa, Homo sapiens
GGKGLGKGGAKR

SEQ ID NO: 4 - protein, 14 aa, Homo sapiens
GKGGKGLGKGGAKR

SEQ ID NO: 5 - protein, 14 aa, Homo sapiens
GKGGKGLGKGGAKR

SEQ ID NO: 6 - protein, 14 aa, Homo sapiens
GKGGKGLGKGGAKR

SEQ ID NO: 7 - protein, 13 aa, Homo sapiens
GKGGKGLGKGGAK

SEQ ID NO: 8 - protein, 13 aa, Homo sapiens
GKGGKGLGKGGAK

SEQ ID NO: 9 - protein, 13 aa, Homo sapiens
GKGGKGLGKGGAK

SEQ ID NO: 10 - protein, 11 aa, Homo sapiens
GGKGLGKGGAK

SEQ ID NO: 11 - protein, 11 aa, Homo sapiens
GGKGLGKGGAK

SEQ ID NO: 12 - protein, 14 aa, Homo sapiens
GKGGKGLGKGGAKR

SEQ ID NO: 13 - protein, 14 aa, Homo sapiens
GKGGKGLGKGGAKR

SEQ ID NO: 14 - protein, 14 aa, Homo sapiens
  misc_feature (2)..(7): Xaa can be any naturally occurring amino acid
CXXXXXXKXXXXXX

SEQ ID NO: 15 - protein, 14 aa, Homo sapiens
  misc_feature (2)..(7): Xaa can be any naturally occurring amino acid
CXXXXXXKXXXXXX

SEQ ID NO: 16 - protein, 14 aa, Homo sapiens
  misc_feature (2)..(7): Xaa can be any naturally occurring amino acid
CXXXXXXKXXXXXX

SEQ ID NO: 17 - protein, 14 aa, Homo sapiens
  misc_feature (2)..(7): Xaa can be any naturally occurring amino acid
CXXXXXXKXXXXXX

SEQ ID NO: 18 - protein, 31 aa, Homo sapiens
SGRGKGGKGLGKGGAKRHRKVKKKKKKGFGG

SEQ ID NO: 19 - protein, 393 aa, Homo sapiens [sequence omitted]

SEQ ID NO: 20 - protein, 102 aa, Homo sapiens
SGRGKGGKGLGKGGAKRHRKVLRDNIQGITKPAIRRLARRGGVKRISGLIYEETRGVLKVFLENVIRDAVTYTEHAKRKTVTAMDVVYALKRQGRTLYGFGG

SEQ ID NO: 21 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 22 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 23 - protein, 393 aa, Homo sapiens [sequence omitted]

SEQ ID NO: 24 - protein, 102 aa, Homo sapiens; identical to SEQ ID NO: 20

SEQ ID NO: 25 - protein, 2414 aa, Homo sapiens; identical to SEQ ID NO: 21

SEQ ID NO: 26 - protein, 2442 aa, Homo sapiens; identical to SEQ ID NO: 22

SEQ ID NO: 27 - protein, 47 aa, Homo sapiens
PEPAKSAPAPKKGSKKAVTKAQKKGSKKRKHAVSEGTKAVTKYTSSK

SEQ ID NO: 28 - protein, 44 aa, Homo sapiens
ARTKQTARKSTGGKAPRKQLATKAARKSAPATGGVKKPHRKREA

SEQ ID NO: 29 - protein, 42 aa, Homo sapiens
SGRGKGGKGLGKGGAKRHRKVLRDNIQGITKPAIRTLYGFGG